(19) United States
(12) Patent Application Publication
(10) Pub. No.: US 2020/0323986 A1
(43) Pub. Date: Oct. 15, 2020

(54) HYDROPHOBIC ARENESULFONATE SALTS

(71) Applicant: Theracaine LLC, San Francisco, CA (US)

(72) Inventor: Samuel P. Sawan, San Rafael, CA (US)

(21) Appl. No.: 16/860,365

(22) Filed: Apr. 28, 2020

Related U.S. Application Data
(63) Continuation of application No. PCT/US2018/058141, filed on Oct. 30, 2018.
(60) Provisional application No. 62/578,861, filed on Oct. 30, 2017.
(60) Provisional application No. 62/764,902, filed on Aug. 16, 2018.
(60) Provisional application No. 62/578,857, filed on Oct. 30, 2017.
(60) Provisional application No. 62/589,108, filed on Nov. 21, 2017.
(60) Provisional application No. 62/589,134, filed on Nov. 21, 2017.

(51) Int. Cl. A61K 47/20; A61K 31/167

(52) U.S. Cl. CPC A61K 47/20; A61K 31/167

(57) ABSTRACT

The invention provides acid addition salts of a basic therapeutic agent wherein the acid is represented by Formula I or Formula II,

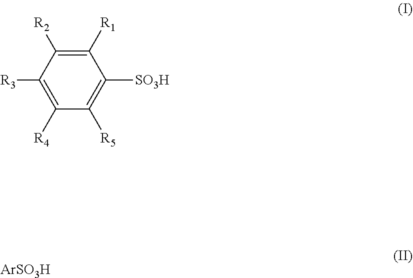

wherein Ar, R₁, R₂, R₃, R₄, and R₅ are as defined herein. The invention also provides pharmaceutical compositions comprising a pharmaceutically acceptable carrier or excipient and an acid addition salt of the invention and a method of using an acid addition salt of the invention for treating a disease or disorder in a subject in need thereof.

### RELATED APPLICATIONS

This application is a continuation of International Application No. PCT/US18/58141, which designated the United States and was filed on Oct. 30, 2018, published in English, which claims the benefit of U.S. Provisional Application No. 62/578,861, filed on Oct. 30, 2017, U.S. Provisional Application No. 62/764,902, filed on Aug. 16, 2018, U.S. Provisional Application No. 62/578,857, filed on Oct. 30, 2017, U.S. Provisional Application No. 62/589,108, filed on Nov. 21, 2017 and U.S. Provisional Application No. 62/589,134, filed on Nov. 21, 2017. The entire teachings of the above application are incorporated herein by reference.

### BACKGROUND OF THE INVENTION

The physicochemical characteristics and economic benefit of a medicinal drug can be manipulated and improved by conversion to a salt form. Selecting the appropriate salt is considered to be a very important step since each salt shows distinctive properties to the parent drug. Usually the salt-forming agents are selected by testing and experience according to the cost of raw materials, simplicity of crystallization and the amount of yield produced.

It has been estimated that approximately 50% of all drug molecules marketed as medicinal products are administered in a form of salts. This simple statistic shows that salt formation of drug substances is a central pre-formulation process and it must be associated with significant advantages. Certainly, many drug molecules are characterized by undesirable physicochemical properties that can be effectively improved by converting a basic or acidic drug into a salt form.

Salt formation offers many advantages to the pharmaceutical products as it can improve the solubility, dissolution rate, permeability and efficacy of the drug. In addition, salts can help in the improvement of the hydrolytic and thermal stability. Salts also play an important role in targeted drug delivery of dosage form (e.g. in the cases of controlled release dosage forms).

In one embodiment salt formation involves, in essence, pairing the parent drug molecule with an appropriate counterion. The essential prerequisite is the presence of a basic functional group in the drug's structure that allow sufficient ionic interaction between the drug and the acid. The charged groups in the structure of the drug and the conjugate base of the acid are attracted by ionic intermolecular forces. At favorable thermodynamic conditions, the salt is precipitated in the crystallized form.

The choice of the salt forming agent is dictated by a number of criteria that the salt is intended to meet. Formulation (dosage form) type may influence this choice—for solid dosage forms, oral solutions, and injectables, highly soluble hydrochlorides and mesylates, besylates and other forms can be chosen. Alternatively, for suspensions or otherwise slow drug release profiles, relatively hydrophobic counter ions may be preferred such as those described herein.

### SUMMARY OF THE INVENTION

The invention provides an acid addition salt of a basic therapeutic agent wherein the acid is of Formula I,

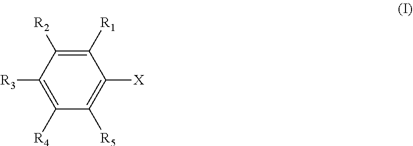

wherein R₁, R₂, R₃, R₄ and R₅ are each independently hydrogen, halogen, C₁-C₁₂-alkyl or halo-C₁-C₁₂-alkyl; and X is —SO₃H, —C(O)OH or —PO(OR₆)(OH), where R₆ is hydrogen or C₁ to C₆-alkyl; provided that at least one of R₁, R₂, R₃, R₄ and R₅ is not hydrogen and further provided that the compounds of Formula I do not include 4-methylbenzenesulfonic acid.

In another embodiment, the invention provides an acid addition salt of a basic therapeutic agent wherein the acid is of Formula II,

Ar—X (II),

wherein Ar is an optionally substituted polycyclic aryl group and X is as defined above.

The invention also provides a pharmaceutical composition comprising an acid addition salt of the invention and a pharmaceutically acceptable excipient or carrier.

The invention further includes methods of treating a disease or disorder in a subject in need thereof, comprising administering to the subject an effective amount of an acid addition salt of the invention.

### BRIEF DESCRIPTION OF THE DRAWINGS

FIG. 1 is a graph of plasma bupivacaine concentration normalized to Cmax versus time following subcutaneous administration of particles of lidocaine 3,5-dibromo-4-methylbenzenesulfonate (L-4/2-650) and lidocaine 4-bromobenzenesulfonate (L-4/1-650) as described in Example 11.

FIG. 2 is a graph of plasma bupivacaine concentration normalized to Cmax versus time following subcutaneous administration of particles of bupivacaine 3,5-dibromo-4-methylbenzenesulfonate (B-4/2-650) and bupivacaine 4-bromobenzenesulfonate (B-4/1-650) as described in Example 11.

FIG. 3 is a graph of plasma lidocaine concentration (ng/mL) versus time following subcutaneous administration of particles of lidocaine 3,5-dibromo-4-methylbenzenesulfonate (L-4/2-650) and lidocaine 4-bromobenzenesulfonate (L-4/1-650) as described in Example 11.

FIG. 4 is a graph of plasma lidocaine concentration (ng/mL) versus time following subcutaneous administration of particles of lidocaine 3,5-dibromo-4-methylbenzenesulfonate (L-4/2-650) and lidocaine 4-bromobenzenesulfonate (L-4/1-650) as described in Example 11.

FIG. 5 is an illustration of a polymeric tube delivery device of the invention.

FIG. 6 is an illustration of a wound dressing comprising polymeric delivery devices.

FIG. 7 is a graph of theoretical drug release over time as a function of the drug surface area for a 5 cm² dressing.

### DETAILED DESCRIPTION OF THE INVENTION

The invention provides acid addition salts of a basic, for example monobasic or polybasic, therapeutic agent wherein the acid is represented by Formula I:

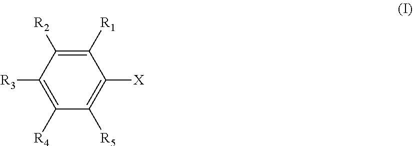

wherein R₁, R₂, R₃, R₄ and R₅ are each independently hydrogen, halogen, C₁-C₁₂-alkyl or halo-C₁-C₁₂-alkyl; and X is —SO₃H, —C(O)OH or —PO(OR₆)(OH), where R₆ is hydrogen or C₁ to C₆-alkyl; provided that at least one of R₁, R₂, R₃, R₄ and R₅ is not hydrogen and further provided that the compounds of Formula I do not include 4-methylbenzenesulfonic acid.

In another embodiment, the invention provides an acid addition salt of a basic therapeutic agent wherein the acid is of Formula II,

Ar—X (II),

wherein Ar is an optionally substituted polycyclic aryl group and X is as defined above. Preferably the polycyclic aryl group is an optionally substituted biphenyl, naphthyl, anthracenyl, or indenyl. Preferably, the number of substituents is 0 to 4. In preferred embodiments, the substituents are independently selected from alkyl, haloalkyl and halogen. In certain embodiments, each substituents is independently selected from methyl, trifluoromethyl and halogen.

In preferred embodiments of the compounds of Formula I and Formula II, X is —SO₃H.

The term “alkyl” is intended herein to include both branched and straight chain, saturated aliphatic hydrocarbon radicals/groups having the specified number of carbons. Preferably, an alkyl group is a C₁-C₂₄ alkyl group, a C₃ to C₂₄ alkyl group or a C₄ to C₂₄-alkyl group. In certain embodiments, an alkyl group is a C₁-C₁₂-alkyl group. Suitable alkyl groups include methyl, ethyl, n-propyl, i-propyl, n-butyl, sec-butyl, isobutyl, t-butyl, n-pentyl, pent-2-yl, pent-3-yl, 3-methylbutyl, 3-methylbut-2-yl, neopentyl, n-hexyl, hex-2-yl, hex-3-yl, 4-methylpentyl, 4-methylpent-2-yl, 3,3-dimethylbutyl, and 3,3-dimethylbut-2-yl. Preferably, the alkyl group is methyl or an n-C₂-C₂₄-alkyl, and more preferably an n-C₃-C₁₄-alkyl, an n-C₃-C₁₀-alkyl, or an n-C₃-C₈-alkyl.

The term “halogen” is intended herein to refer to fluorine, chlorine, bromine or iodine. Preferred halogens are fluorine, chlorine and bromine.

The term “haloalkyl group” is intended herein to refer to an alkyl group in which at least one hydrogen atom is substituted with a halogen atom, preferably a fluorine, chlorine or bromine atom. Preferred haloalkyl groups have at least two or three halogen substituents. In a haloalkyl having two or more halogen substituents, the halogen substituents can be the same or different. A “perhaloalkyl” group is a haloalkyl group in which all hydrogen atoms are substituted with halogen atoms, preferably chlorine and/or fluorine atoms. Preferably, a perhaloalkyl group is a perchloroalkyl group or a perfluoroalkyl group, more preferably a perfluoroalkyl group.

The term “polycyclic aryl group” is intended herein to a moiety comprising two or more aryl groups which are connected by a single bond or fused. Suitable polycyclic aryl groups include biphenyl, naphthyl, anthracenyl, phenanthryl and 1-H-phenalenyl. Preferred polycyclic aryl groups include biphenyl and naphthyl.

In certain embodiments of the compounds of Formula I, at least one of R₁ to R₅ is a halogen. In certain embodiments, two, three, four or five of R₁ to R₅ are halogen. In compounds having more than one halogen, the halogens can be the same or different. In certain embodiments, at least two of R₁ to R₅ is halogen and the halogens are the same. Preferred halogens include fluorine, chlorine and bromine. In certain embodiments, each of R₁ to R₅ is independently a halogen. In this embodiment, R₁ to R₅ are preferably the same halogen. In certain embodiments,

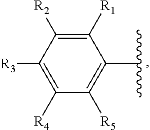

is pentafluorophenyl, pentachlorophenyl or pentabromophenyl.

In certain embodiments of the compounds of Formula I, at least one of R₁ to R₅ is an alkyl group. In certain embodiments, two, three, four or five of R₁ to R₅ are alkyl groups. In compounds having more than one alkyl groups, the alkyl groups can be the same or different. In certain embodiments, at least two of R₁ to R₅ are the same alkyl group.

In certain embodiments of the compounds of Formula I, at least one of R₁ to R₅ is a haloalkyl group. In certain embodiments, two, three, four or five of R₁ to R₅ are haloalkyl groups. In compounds having more than one haloalkyl groups, the haloalkyl groups can be the same or different. In certain embodiments, at least two of R₁ to R₅ are the same haloalkyl group.

In certain embodiments of the compounds of Formula I, at least one of R₁ to R₅ is a halogen and at least one of the others is an alkyl or haloalkyl group. In certain embodiments R₁ to R₅ include at least one halogen, such as fluorine, chlorine or bromine, and at least methyl. In certain embodiments, at least one of R₁ to R₅ is halogen and the remainder are independently hydrogen or methyl; in these embodiments, one or two of R₁ to R₅ is halogen, preferably chloro or bromo, one of R₁ to R₅ is methyl or hydrogen and the remainder are hydrogen. In certain embodiments, one of R₁ to R₅ is methyl, two of R₁ to R₅ are halogen, preferably chloro or bromo, and the remainder are hydrogen.

In certain embodiments of the compounds of Formula I, at least one of R₁ to R₅ is hydrogen. Preferably, at least two or at least three of R₁ to R₅ are hydrogen. In certain embodiments, four of R₁ to R₅ are hydrogen.

In certain embodiments, the compound of Formula I is selected from the compounds below.

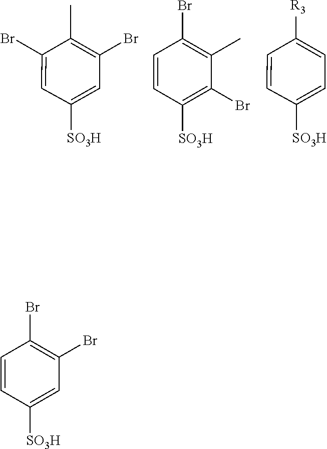

In certain embodiments, R₃ is bromo, alkyl or haloalkyl. In other embodiments, R₃ is halo, preferably bromo or chloro, C₃-C₂₄-alkyl or C₆-C₂₀ alkyl. In certain embodiments, R₃ is chloro, bromo or n-C₁₀-C₂₄-alkyl, such as n-C₁₄-alkyl or n-C₂O-alkyl.

In other embodiments, the compound of Formula I is selected from the compounds below.

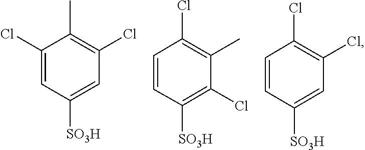

In certain embodiments, the compound of Formula I or Formula II is selected from 4-fluoro-2,6-dimethylbenzene-1-sulfonic acid; naphthalene-1-sulfonic acid; 3,5-difluoro-4-methylbenzene-1-sulfonic acid; 2,4,6-trimethylbenzene-1-sulfonic acid; [1,1′-biphenyl]-4-sulfonic acid; 4-chloro-2,6-dimethylbenzene-1-sulfonic acid; 5-chloronaphthalene-1-sulfonic acid; 2,6-dimethyl-4-(trifluoromethyl)benzene-1-sulfonic acid; 4-bromo-2,6-dimethylbenzene-1-sulfonic acid; 5-bromonaphthalene-1-sulfonic acid; 2,6-dimethyl-4-(trichloromethyl)benzene-1-sulfonic acid; 4-iodo-2,6-dimethylbenzene-1-sulfonic acid; 4′-chloro[1,1′-biphenyl]-4-sulfonic acid; 5-iodonaphthalene-1-sulfonic acid; 3,5-dichloro-4-methylbenzene-1-sulfonic acid; 2,4,6-trichloro-3,5-dimethylbenzene-1-sulfonic acid; 4′-bromo[1,1′-biphenyl]-4-sulfonic acid; 4′-iodo[1,1′-biphenyl]-4-sulfonic acid; 3,5-dibromo-4-methylbenzene-1-sulfonic acid; 3,5-dichloro-4-methylbenzene-1-sulfonic acid; 3,5-diiodo-4-methylbenzene-1-sulfonic acid; 4-bromo-3,5-bis(trifluoromethyl)benzene-1-sulfonic acid; 2,3,4,5,6-pentachlorobenzene-1-sulfonic acid; 2,3,4,5,6-pentafluorobenzene-1-sulfonic acid; 2,4,6-triethylbenzene-1-sulfonic acid; 4-bromo-3,5-bis(trichloromethyl)benzene-1-sulfonic acid; 2,6-dimethyl-4-(tribromomethyl)benzene-1-sulfonic acid; 2,4,6-tri(trichloromethyl)benzene-1-sulfonic acid; 2,4,6-tri(trifluoromethyl)benzene-1-sulfonic acid; 4-iodo-3,5-bis(trifluoromethyl)benzene-1-sulfonic acid; 2,6-dimethyl-4-(triiodomethyl)benzene-1-sulfonic acid; 2,4,6-tripropylbenzene-1-sulfonic acid; 2,4,6-tri(tribromomethyl)benzene-1-sulfonic acid; and 2,4,6-tri(trifluoromethyl)benzene-1-sulfonic acid.

The term “basic therapeutic agent”, which is used interchangeably herein with the term “basic drug” or just “drug”, refers to a drug which contains one or more basic functional groups. Basic therapeutic agents include monobasic therapeutic agents, which contain only one basic functional group under the conditions of salt formation, and polybasic therapeutic agents, which contain at least two such functional groups. Basic functional groups include primary, secondary, tertiary and quaternary amino groups, amidino groups, amino groups, guanidino groups and basic N-containing heteroaryl groups.

In certain embodiments, the acid addition salt of the invention is represented by Formula III:

B(H)ₘ₊ₙYₘZₙ (III)

where B is a basic drug, and Y is the conjugate base of a compound of Formula I or Formula II, that is Y is Ar—W or

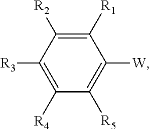

wherein W is SO₃⁻, C(O)O⁻ or —PO(OR₆)O⁻;
is a pharmaceutically acceptable monoanion other than Y; and m+n is the number of basic groups on B, provided that m is at least 1. Preferably m+n is 1, 2, or 3. Preferred acid addition salts are represented by Formula IV,

B(H)ₘYₘ (IV)

where m is the number of basic groups on B, preferably 1, 2 or 3. In particularly preferred embodiments, B is a monobasic drug (i.e., m is 1) and the acid addition salt of the invention is represented by Formula V,

B(H)Y (V).

In the acid addition salts of Formulas III, IV and V, W is preferably —SO_{3″.}

It is to be understood that a quaternary ammonium functional group carries a positive charge without protonation. Thus, in a basic drug which has such a group, the overall positive charge on the drug compound will be greater than the number of protonated sites. For example, in salts of Formula V in which the basic drug has a single basic functional group which is a quaternary ammonium group, there is no additional proton present and the formula is BY.

In certain embodiments, the conjugate base of an acid of Formula I or Formula II has relatively low surface activity or surfactancy. In certain embodiments, the conjugate base of an acid of Formula I or Formula II has a critical micelle concentration (“CMC”) in water at 1 atmosphere and 25° C. which is greater than 20 mM. In certain embodiments, the CMC is greater than 30 mM, 40 mM or 50 mM. In other embodiments, the CMC is greater than 70 mM, 90 mM, 100 mM, 125 mM, 150 mM, 175 mM, 200 mM or 225 mM.

In certain embodiments, the acid of Formula I or Formula II has a Log P value of 1 or greater, for example, 2 or greater, 3 or greater, 4 or greater or 5 or greater, as calculated using ACD Labs software. This approach to calculating Log P employs a Classic model, which relies on the separation of the molecule in question into its constituent parts and summing those values as determined for sample compounds that have been tabulated from the literature.

Suitable basic drugs are set forth as follows: Analgesics (opioids) and codeine derivatives such as morphine, benzylmorphine, propoxyphene, methadone, pentazocine, sufenatanil, alfentanil, fentanyl, pethidine, butorphanol, buprenorphine, diamorphine, dihydrocodeine, dypyrone, oxycodone, dipipanone, alphaprodine, levorphanol, dextromoramide, hydromorphone, nalbuphine, oxymorphone, hydrocodone, nalorphine (antagonist), naloxone (antagonist); Antimicrobials including quinolones such as norfloxacin, ciprofloxacin, lomefloxacin, balofioxacin, ofloxacin, sparfloxacin, tosufloxacin, temafloxacin, clinafloxacin, perfloxacin, tosufloxacin, enoxacin, amifloxacin, fleroxacin; Antimicrobials including aminoglycosides such as streptomycin, amikacin, gentamicin, tobramycin, neomycin, josamycin, spectinomycin, kanamycin, framycetin, paromomycin, sissomycin, viomycin; Glycopeptides such as vancomycin; Lincosamides such as clindamycin, lincomycin; Penicillins such as cephalosporins and cefepime, related β-lactams, cefmenoxime, cefotiam, cephalexin, bacampicillin, lenampicillin, pivampicillin, talampicillin; Macrolides such as erythromycin, oleandomycin; Tetracyclines such as tetracycline, minocycline, rolitetracycline, methacycline, meclocycline; Antimycobacterials such as isoniazid, pyrimethamine, ethambutol, Antivirals such as acyclovir, saquinavir, indinavir, ganciclovir, amantadine, moroxydine, rimantidine, famciclovir, zalcitabine, cidofovir, valacyclovir, lamivudine, nevirapine; Antiprotozoals such as metronidazole, temidazole, pentamidine, mepacrine, carnidazole, robenidine, emetine, dihydroemetine, halofuginone, homidium, melarsoprol; Antiseptics such as aminacrine; Antifungals such as ketoconazole, itraconazole, miconazole, econazole, clotrimazole, amphotericin B, butoconazole, chlormidazole, croconazole, diamthazole, fenticonazole, nystatin, cloconazole, econazole, miconazole, tioconazole; Anti-depressants such as clomipramine (all classes), lofepramine, phenelzine, tranylcypromine, dothiepin, nortryptaline, amitryptaline, imipramine, mianserin, maprotiline, desipramine, trazodone, fluoxetine, trimipramine, citalopram, doxepin, fluvoxamine, lofepramine, nomifensine, paroxetine, Anti-diabetics such as glipizide, metformin, phenformin; Anti-convulsants such as carbamazepine, ethosuxamide, diphenylhydantoin, phenytoin(-OH), primidone, methsuximide; Anticholinergics such as atropine (antimuscarinics), benztropine (all classes), scopolamine, homatropine, hyoscine, hyoscyamine, orphenadrine, pirenzipine, procyclidine, telenzipine, propantheline, dicyclomine, biperiden, trihexphenidyl, oxybutinin, benzhexol, biperiden, ipratropium, pipenzolate, mepenzolate, cyclopentolate; Anthelminitics such as albendazole, mebendazole, flubendazole, fenbendazole, pyrantel, ivermectin; Antigout such as allopurinol, colchicine; Antihistamines and chlorpheniramine phenothiazines such as dimenhydrinate (all classes), hydroxyzine, diphenhydramine, bromodiphenhydramine, astemizole, loratidine, acepromazine, thioridazine, brompheniramine, carbinoxamine, chlorcyclizine, chloropyramine, chlorphentermine, chlorprothixene, dexchlorpheniramine, antazoline, azatidine, azalastine, clemastine, clemizole, cyroheptadine, diphenylpyraline, doxylamine, flunarizine, mequitazine, meclozine, mepyramine, pheniramine, terfenadine, triprolidine, trimeprazine, ebastine, cinnarizine; Anti-migraines such as ergotamine, dihydroergotamine, methysergide, sumatriptan, naritriptan, almotriptan, zolmitriptan, rizatriptan, eletriptan, flumedroxone, pizotifen; Anti-tussives and dextromethorphan mucolytics such as pholcodeine, acetylcysteine, noscapine; Antineoplastics and azathiprine Immunosupressants such as methyluracil, fluorouracil, vincristine, vinblastine, melphalan, cyclophosphamide, aminoglutethimide, mercaptopurine, tamoxifen, chlorambucil, daunorubicin, mechlorethamine, doxorubicin; Anti-malarials such as quinine, chloroquine, pyrimethamine, amodiaquine, piperaquine, proguanil, chloroproguanil, mefloquine, primaquine, halofantrine; Anxiolytics, and Sedatives such as bromazepam; Hypnotics, and Antipsycotics such as nitrazepam, diazepam, oxazepam; Benzodiazepines such as clonazepam, chlorazepate, lorazepam, midazolam, triazolam, flunitrazepam; Butyrophenones such as droperidol, haloperidol; Barbiturates such as allobarbitone, aprobarbitone, phenobarbitone, amylobarbitone, barbitone, butobarbitone, zopiclone, hydroxyzine, buspirone, tandospirone, Bronchodilators such as theophylline; Cardiovascular Drugs including β-Blockers such as acebutatol, alprenolol, atenolol, labetalol, metopralol, nadolol, timolol, propanolol, pindolol, tolamolol, sotalol, oxprenolol, bunitrolol, carazolol, indenolol; Cardiovascular Drugs including Anti-arrythmics/cardiotonics such as disopyramide, cardiotonics, mexilitine, tocainide, aprindine, procainamide, quinidine, dobutamine; Cardiovascular Drugs including Ca channel blockers (all classes) including verapamil, diltiazem, amlodipine, felodipine, nicardipine, gallopamil, prenylamine; Cardiovascular Drugs including Antihypertensives/Vasodilators including diazoxide, guanethidine, clonidine, hydralizine, dihydralizine, minoxidil, prazosin, phenoxybenzamine, reserpine, phentolamine, perhexiline, indapamide, debrisoquine, bamethan, bethanidine, dobutamine, indoramin; Cardiovascular Drugs including Ace inhibitors captopril, enalapril, lisinopril, ramipril, imidapril; CNS stimulants/anorectics including methylphenidate, fenfluramine, amphetamine, methamphetamine, bemegride, caffeine, dexamphetamine, chlorphentamine, fencamfamine, prolintane; Diuretics such as furosemide, acetazolamide, amiloride, triampterene, bendrofluazide, chlorothiazide, chlorthalidone, cyclothiazide, hydroflumethiazide, hydrochlorothiazide, hydroflumethiazide; Gatrointestinal Agents including Motility enhancers, modulators and anti-emetics such as domperidone; metoclopramide; cisapride, prochlorperazine, pirenzipine, cinitapride, cyclizine, chlorpromazine, prochloperazine, promethazine; Gastrointestinal Agents including Acid secretion modulators such as cimetidine, ranitidine, famotidine, omeprazole, nizatidine; Gatrointestinal Agents including Anti-diarrheals, including loperamide, diphenoxylate; Gatrointestinal Agents including emetics such as apomorphine; Muscle relaxants such as chlorzoxazon, rocuronium, suxamethonium, vecuronium, atracurium, fazadinium, doxacurium, mivacurium, pancuronium, tubocurarine, pipecurium, decamethonium, tizanidine, piridinol, succinylcholine, acetylcholine; Cholinergic Agents such as benzpyrinium, edrophonium, physostigmine, neostigmine, pyridostygmine; β-adrenergic agonists such as adrenaline ephedrine, pseudoephedrine, amidephrine, oxymetazoline, xylometazoline, terbutaline, salbutamol, salmeterol, phenylpropanolamine, cyclopentamine, phenylephrine, isoproterenol, fenoterol, xamoterol; Other CNS active agents such as dopamine, levodopa; Endocrine agents such as bromocriptine, propylthiouracil; Local anesthetics such as lidocaine (lignocaine), procaine, amethocaine, bupivacaine, butacaine, oxybuprocaine, mepivacaine, cocaine, prilocaine, amylocaine, chloroprocaine, cinchocaine, etidocaine, propoxycaine, tropacocaine, ropivacaine; Miscellaneous Mydriatics such as cyclopentolate, methazolamide, dorzolamide, acetazolamide, dynorphins, enkephalins, oxytocin and vasopressin. Additional basic therapeutic agents include naltrexone, varenicline, bacitracin, linezolid, daptomycin, granisetron, ondansetron, aripiprazole, risperidone, olanzapine, clozapine, thorazine, ipratropium, and bethanecol.

Preferably, the basic therapeutic agent is a local anesthetic such as, but not limited to: lidocaine (lignocaine), procaine, amethocaine, bupivacaine, butacaine, oxybuprocaine, mepivacaine, cocaine, prilocaine, amylocaine, chloroprocaine, cinchocaine, etidocaine, propoxycaine, tropacocaine, and ropivacaine. Preferably the basic therapeutic agent is lidocaine, bupivacaine or ropivacaine. More preferably the basic therapeutic agent is bupivacaine or lidocaine, and most preferably bupivacaine.

The acid addition salts of basic therapeutic agents in accordance with the present invention provide, among other advantages, sustained or extended therapeutic levels of the therapeutic compound following administration. “Sustained release” typically refers to shifting bioavailability of the drug toward pseudo first-order release kinetics or to other release profiles based upon how particles may aggregate in vivo. Sustained release may be due to several factors including, but not limited to, the decreased solubility of the acid addition salt relative to the parent drug. The term “sustained release” as used herein means that administration of an acid addition salt of a basic therapeutic agent of the invention to a subject results in effective systemic, local or plasma levels of the parent basic therapeutic agent in the subject's body for a period of time that is longer that resulting from administration of the parent basic therapeutic agent which is not formulated as the acid addition salt of the present invention.

The choice of acid of Formula I or Formula II can be used to selectively control the hydrophobicity and aqueous solubility of the resulting salt of any given basic therapeutic agent and thereby control the release rate of the drug.

In certain embodiments, the basic drug and the acid do not form a true salt, i.e., an ionic compound. For example, a weak acid and a weak base can, under certain conditions, for a mixture, alloy or eutectic. Without being bound by theory, it is believed that in certain combinations of weakly basic drugs and weak acids, a mixture of the two results in which proton transfer from the acid to the base is incomplete. Such combinations can provide extended duration of release of the drug similar to salts when compositions are properly selected.

In a preferred embodiment, a compound of the invention provides sustained delivery of the parent drug over hours, days, weeks or months when administered, for example, topically, orally or parenterally, to a subject. For example, when delivered parenterally, the compounds can provide sustained delivery of the drug for up to 1, 7, 15, 30, 60, 75 or 90 days or longer. Without being bound by theory, it is believed that the compounds of the invention form an insoluble depot upon parenteral administration, for example by subcutaneous, intramuscular or intraperitoneal injection.

The pharmaceutical compositions of the present invention comprise a therapeutically effective amount of an acid addition salt of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; cyclodextrins such as alpha-(α), beta-(β) and gamma-(γ) cyclodextrins; starches such as corn starch and potato starch; cellulose and its derivatives such as sodium carboxymethylcellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In certain embodiments, the formulations include a viscoelastic polymer, such as hyaluronic acid, chondroitin sulfate or a glycosaminoglycan. In other embodiments, the formulations include a water soluble low molecular weight polymer, such as polyethylene glycol.

The pharmaceutical compositions of this invention may be administered orally, parenterally, by inhalation spray, topically, rectally, nasally, buccally, vaginally or via an implanted reservoir. In a preferred embodiment, administration is parenteral administration by injection.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intracisternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, dimethylacetamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable suspension or emulsion, such as INTRALIPID®, LIPOSYN® or OMEGAVEN®, or solution, in a nontoxic parenterally acceptable diluent or solvent, for example, as a solution in 1,3-butanediol. INTRALIPID® is an intravenous fat emulsion containing 10-30% soybean oil, 1-10% egg yolk phospholipids, 1-10% glycerin and water. LIPOSYN® is also an intravenous fat emulsion containing 2-15% safflower oil, 2-15% soybean oil, 0.5-5% egg phosphatides 1-10% glycerin and water. OMEGAVEN® is an emulsion for infusion containing about 5-25% fish oil, 0.5-10% egg phosphatides, 1-10% glycerin and water. Among the acceptable vehicles and solvents that may be employed are water, Ringer's solution, USP and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The formulations can also be sterilized by other methods, including heat and/or radiation, such as gamma, ultraviolet or electron beam radiation.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43650 by Montgomery, all of which are incorporated herein by reference). A discussion of pulmonary delivery of antibiotics is also found in U.S. Pat. No. 6,014,969, incorporated herein by reference.

In preferred embodiments, the compounds of the invention, or pharmaceutical compositions comprising one or more compounds of the invention, are administered parenterally, for example, by intramuscular, subcutaneous or intraperitoneal injection. Without being bound by theory, it is believed that upon injection, salts of the invention form an insoluble or sparingly soluble depot from which drug molecules are released over time.

By a “therapeutically effective amount” of a drug compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect).

As used herein, the term “effective amount of the subject compounds,” with respect to the subject method of treatment, refers to an amount of the subject compound which, when delivered as part of a desired dose regimen, brings about management of the disease or disorder to clinically acceptable standards.

“Treatment” or “treating” refers to an approach for obtaining beneficial or desired clinical results in a patient. For purposes of this invention, beneficial or desired clinical results include, but are not limited to, one or more of the following: alleviation of symptoms, diminishment of extent of a disease, stabilization (i.e., not worsening) of a state of disease, preventing spread (i.e., metastasis) of disease, preventing occurrence or recurrence of disease, delay or slowing of disease progression, amelioration of the disease state, and remission (whether partial or total).

In certain embodiments, the salts of the invention are provided in the form of particles.

Preferably, the invention provides anesthetic particles for the treatment of pain due to an injury, particularly a wound, where the particles comprise as their major ingredient an acid addition salt of the invention where the therapeutic agent is a local anesthetic, such as a “caine” anesthetic. Local anesthetics of the “caine” family are weak monobases. (by “caine” is intended anesthetics that end in the suffix “caine”, which in certain embodiments include an amino acid amide or ester). One of the classes of caine anesthetics are amine bases and also include an aromatic ring, for example, a meta-xylyl group, and an amide or ester functionality. The aromatic group with the other entities results in hydrophobicity, so that the members of the class are frequently employed as their hydrochloride salts to allow for water solubility. Examples of such anesthetics of the caine family include lidocaine (lignocaine), procaine, bupivacaine, ropivacaine, butacaine, oxybuprocaine, mepivacaine, prilocaine, amylocaine, chloroprocaine, etidocaine, propoxycaine and tropacocaine. Caines of particular interest are lidocaine, bupivacaine and ropivacaine.

In certain embodiments, the salts of the invention, such as the caine salts, are provided in the form of particles. In certain embodiments, the particles consist of one or more caine salts of Formula V, or consist essentially of one or more caine salts of Formula V. In certain embodiments, the particles can have a 1:1 equivalent ratio of the drug to the acid or one of the components may be in excess, usually not more than about 5-fold excess, generally up to about 0.5, or up to about a 0.2, equivalent excess of either of the components of the salt may be present. In certain embodiments, the particles include excess acid. By having excess acid, the release rate of the drug, such as the caine, from the salt may be diminished by virtue of the common ion effect, where the dissolution of the excess acid will act to slow or retard the dissolution rate of the drug salt compound in the particles. In other embodiments, the particles include a caine salt of Formula V, as described herein, and a second caine salt of Formula V, wherein preferably the two caine salts have different aqueous solubilities.

The particles can further comprise two or more caine salts of the invention, differing in either or both of the caine agent and the acid. For example, the particles can comprise two or more caine salts of Formula (V) in which the anion is different and which differ in hydrophobicity. By using different acid addition salts, the rate of release of the anesthetic can be modulated, with acids with smaller R groups usually providing for more rapid release. The composition may be a mixture of different sized particles, usually comprising not more than two different distributions, where each of the different distributions has at least about 75% of the weight of the particles within 50%, more usually within 25%, of the median weight. The median weights of the two differently sized compositions will usually differ by at least about 25%, more usually at least about 50% and there may be a two-fold difference or greater. In this way both composition and particle size can be varied to provide the optimum release profile for the particular application for the subject compositions.

In one embodiment, the composition comprises particles of a caine salt of Formula V and a soluble salt of the caine or a different caine. The soluble caine salt can be in a solid form, for example, in the form of particles, or in solution. In one embodiment, the particles of the caine salt of Formula V are suspended in a solution comprising the soluble caine salt. The solution can be an aqueous solution or a solution of a pharmaceutically acceptable hydrophilic organic solvent. The soluble caine salt is preferably the hydrochloride, hydrobromide, acetic acid or nitric acid salt, preferably the hydrochloride salt. For example, the composition can comprise a salt of lidocaine, bupivacaine or ropivacaine with an acid of Formula I or Formula II and a soluble salt of one of these caines, such as lidocaine hydrochloride, bupivacaine hydrochloride or ropivacaine hydrochloride. Preferably, the same caine is present in both salts. Such compositions provide both a rapid onset of action due to the soluble salt and sustained action due to the caine salt of Formula V.

The particles can further comprise one or more pharmaceutically acceptable excipients or additives, such as surfactants, polymers and salts. Preferably, the particles do not include a matrix, such as polymer matrix, which prolongs release of the drug.

The size distribution of a particle composition of the salts of the invention will generally have at least about 50 weight % within 75%, more usually within 50%, and desirably within 25% of the median size. The median size will generally range from about 1 to about 2000 μm, more usually from about 5 to 1500 μm, desirably from about 5 μm to 1200 μm. Individual compositions of interest have median sizes of about 1 to 25 μm; 5 to 100 μm; 100 to 200 μm, 300 to 500 μm, 500 to 750 μm, 600 to 700 μm and 750 to 1200 μm. In one embodiment, the median size of the particles is about 625 to 675 μm, or about 650 μm.

Depending upon the manner in which the particles are made, they can comprise less than about 2, more usually less than about 1, weight % of the solvent used in their preparation, and preferably undetectable amounts.

The present invention additionally provides compositions comprising salt particles of the invention, for example caine salts, and at least one wetting agent. The compositions can be used to deliver the drug particles to a subject in need of treatment with the drug.

The wetting agent is an excipient which prevents or inhibits aggregation of the particles. Suitable wetting agents include nonionic, amphoteric and ionic wetting agents, such as polyhydroxy compounds, including saccharides and sugar alcohols; polyethers, including polyethylene glycols (PEGs) and polypropylene glycols; and non-ionic surfactants, such as poloxamers. Examples of wetting agents include polysorbate, sorbitan esters, sorbitol, propylene glycol, and poloxamers. Preferred wetting agents include polyethylene glycols having a molecular weight from about 100 amu to about 10,000 amu or from about 100 amu to about 1,000 amu. The PEG can be linear or branched. A particularly preferred polyethylene glycol is PEG200. In certain embodiments, the wetting agent is selected to be soluble in the liquid vehicle. In certain embodiments, the wetting agent is a solid under conditions of formulation and use. In certain embodiments, the wetting agent is a solid under conditions of formulation, but melts at physiological temperature. The amount of wetting agent in the composition is preferably sufficient to substantially inhibit aggregation of the particles.

In certain embodiments, the salt particles are suspended in a liquid wetting agent. In another embodiment, the particles are suspended in a vehicle, such as a liquid, paste, lotion or gel. Suitable vehicles include, but are not limited to water, propylene glycol, polyethylene glycols, polypropylene glycols and mixtures thereof. The vehicle can also be an aqueous solution, such as an aqueous buffer, normal saline or buffered saline. Preferably, not more than about 10 weight %, and usually not more than 5 weight %, of the hydrophobic drug will be soluble in the vehicle; preferably the salt is substantially insoluble in the medium.

In preferred embodiments, the salt is substantially insoluble in the liquid vehicle and the wetting agent is soluble in the liquid vehicle. Preferably, the salt particles are suspended in a solution of the wetting agent in the vehicle.

In certain embodiments, the hydrophobic drug particles are coated with the wetting agent or agents before they are suspended in the vehicle.

In certain embodiments, the salt particles are mixed with a solid wetting agent. Preferably, the solid wetting agent is in the form of particles. More preferably, the size of the wetting agent particles is substantially the same as the size of the salt particles. The solid wetting agent can be any wetting agent which is a solid at room temperature, i.e., at about 25° C. or at physiological temperature, i.e. about 37° C. In one embodiment, the wetting agent is a solid under conditions of formulation, storage and administration, but melts following administration. In another embodiment, the wetting agent remains a solid after administration. In certain embodiments, the solid wetting agent is a solid polyethylene glycol, such as a PEG having a molecular weight of about 1000 amu or greater, preferably from about 1000 amu to about 10,000 amu, and more preferably about 2500 amu to about 7500 amu. In one embodiment, the PEG can have a molecular weight of about 3000 amu to about 3500 amu, or about 3350 amu. In another embodiment, the PEG has a molecular weight of about 5000 to 7000 amu, or about 6000 amu.

The particles of the salt and the particles of the wetting agent can be mixed in any suitable ratio. In certain embodiments, the weight ratio of drug particles to wetting agent particles is from 1/3 to 9.5/1, or about 1/2 to about 9/1. In another embodiment, the ratio is from about 1/1 to about 9/1.

The acid addition salts of local anesthetics of the invention are particularly useful for the treatment of pain. In certain embodiments, the pain is due to a wound, such as a wound due to trauma or surgery. In one embodiment, the salts are useful for the topical treatment of a wound, for example, a surface wound resulting from trauma or surgery. In treating the wound, the particles can be administered directly into the wound bed and onto the tissue for an open wound, for example. The particles can be administered by spraying, coating, painting, injecting, irrigating, adhered to a substrate, which substrate is placed in the wound, or the like. Spraying may be employed for administration of the particles with or without a vehicle, using a pharmacologically acceptable propellant. Air may be pumped to disseminate the particles.

Suitable topical vehicles, vehicles for aerosols and other components for use with the caine salts of the present invention are well known in the art. These vehicles may contain a number of different ingredients depending upon the nature of the vehicle, the nature of the wound, the manner of administration, and the like. The vehicles will provide for a convenient method of administration to the wound, while not adversely affecting the controlled release of the anesthetic from the particles.

Most common propellants are mixtures of volatile hydrocarbons, typically propane, n-butane and isobutane, or hydrofluoroalkanes (HFA): either HFA 134a (1,1,1,2-tetrafluoroethane) or HFA 227 (1,1,1,2,3,3,3-heptafluoropropane) or combinations of the two or compressed gases such as nitrogen, carbon dioxide, air and the like. One may also use a simple air brush means of dispensing the particles where there is literally no solvent but air is drawn and used to dispense the particles.

Liquid media used for dispersing the particles are preferably highly volatile or miscible with the aqueous environment of the wound and rapidly evaporate or dissipate under the conditions of administration. The liquids will for the most part be non-solvents for the anesthetic salt, although there may be minimal solubility. Such vehicles may include non-solvent liquid media that include water, mixtures of water and organic solvents and mixtures of organic solvents. Other additives may include protein-based materials such as collagen and gelatin; silicone-based materials; stabilizing and suspending agents; emulsifying agents; and other vehicle components that are suitable for administration to the skin, as well as mixtures of these components and those otherwise known in the art. The vehicle can further include components adapted to improve the stability or effectiveness of the applied formulation, such as preservatives, antioxidants, and skin penetration enhancers. Examples of such components are described in the following reference works hereby incorporated by reference: Martindale, The Extra Pharmacopoeia (Pharmaceutical Press, London 1993) and Martin (ed.), Remington's Pharmaceutical Sciences.

The choice of a suitable vehicle will depend on the particular physical form and mode of delivery that the formulation is to achieve. Examples of suitable forms include liquids; solids and semisolids such as gels, foams, pastes, creams, ointments, powders and the like; colloidal drug delivery systems, for example, liposomes, microemulsions, microparticles, or other forms.

The topical formulations of the caine salts of the invention can be prepared in a variety of physical forms. For example, solid particles, pastes, creams, lotions, gels, and liquids are all contemplated by the present invention. A difference between these forms is their physical appearance and viscosity, which can be governed by the presence and amount of emulsifiers and viscosity adjusters present in the formulation. Particular topical formulations can often be prepared in a variety of these forms. Solids are generally firm and will usually be in particulate form; solids optionally can contain liquids, emulsifiers, moisturizers, emollients, fragrances, dyes/colorants, preservatives and other active ingredients that increase or enhance the efficacy of the final product. Creams and lotions are often similar to one another, differing mainly in their viscosity; both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams may also contain liquids, emulsifiers, moisturizers, emollients, fragrances, dyes/colorants, preservatives and other ingredients that increase or enhance the efficacy of the final product. Liquids are thinner than creams, lotions, or gels and often do not contain emulsifiers.

Suitable emulsifiers for use in the caine addition salt formulations of the present invention include, but are not limited to ionic emulsifiers, behentirmonium methosulfate, cetearyl alcohol, non-ionic emulsifiers like polyoxyethylene oleyl ether, PEG-40 sterate, ceteareth-12, ceteareth-20, ceteareth-30, ceteareth alcohol, PEG-100 stearate, glyceryl stearate, or combinations or mixtures thereof.

Suitable viscosity adjusting agents for use in the caine salt formulations of the present invention include, but are not limited to protective colloids or non-ionic gums such as hydroxyethyl cellulose, xanthan gum, magnesium aluminum silicate, silica, microcrystalline wax, beeswax, paraffin, and cetyl palmitate, or combinations or mixtures thereof.

Suitable liquids for use in the caine salt formulations of the present invention will be selected to be non-irritating and include, but are not limited to water, propylene glycol, polyethylene glycols, polypropylene glycols and mixtures thereof. Not more than about 10 weight %, usually not more than 5 weight %, of the anesthetic salt will be soluble in the medium; preferably the anesthetic salt will be insoluble in the medium.

Suitable surfactants for use in the caine salt formulations of the present invention include, but are not limited to nonionic surfactants. For example, dimethicone copolyol, polyethylene glycols, including higher PEGs, such as PEG200, polysorbate 20, polysorbate 40, polysorbate 60, polysorbate 80, lauramide DEA, cocamide DEA, and cocamide MEA, are contemplated for use with the formulations of the present invention. In addition, combinations or mixtures of these surfactants can be used in the formulations of the present invention.

Suitable preservatives for use in the caine salt formulations of the present invention include, but are not limited to antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. In addition, combinations or mixtures of these preservatives can be used in the formulations of the present invention.

Suitable moisturizers for use in the caine salt formulations of the present invention include, but are not limited to lactic acid and other hydroxy acids and their salts, glycerin, propylene glycol, and butylene glycol. Suitable emollients include lanolin alcohol, lanolin, lanolin derivatives, cholesterol, petrolatum, lipids, phospolipids, isostearyl neopentanoate and mineral oils. In addition, combinations or mixtures of these moisturizers and emollients can be used in the formulations of the present invention.

Other suitable additional ingredients that may be included in the caine salt formulation of the present invention include, but are not limited to, abrasives, absorbents, anticaking agents, anti-foaming agents, anti-static agents, astringents, binders/excipients, buffering agents, chelating agents, film forming agents, conditioning agents, opacifying agents, pH adjusters and protectants. Examples of each of these ingredients in topical product formulations, can be found in publications by The Cosmetic, Toiletry, and Fragrance Association (CTFA). See, e.g., CTFA Cosmetic Ingredient Handbook, 2^{nd} edition, eds. John A. Wenninger and G. N. McEwen, Jr. (CTFA, 1992).

In certain embodiments, a wetting agent as described above is administered to the wound bed prior to administration of the caine salt particles. For example, a wetting agent or a solution thereof can be applied to the wound bed, followed by administration of the salt particles. The salt particles can be administered immediately following the wetting agent or a period of time, such as a few minutes, for example about 1 to 5 minutes after administration of the wetting agent. Alternatively the wetting agent can be applied singularly to the wound bed to provide the desired effect. In a preferred embodiment, the wetting agent is a polyethylene glycol, such as PEG 200.

In many instances it may be desirable that the health care professional administering the particle formulation is able to insure uniform coverage or otherwise be able to see what areas have been covered and how extensively the particle formulation has been distributed. Therefore, one may include a detectable composition with the particles so that they can be visualized. This may include colored compounds or dyes, fluorescent compounds and even luminescent compounds. The dyes should be highly colored and visible in the presence of blood, while the fluorescent compounds should fluoresce under ultra-violet light. See, for example, Richard P. Haugland; Molecular Probes—Handbook of Fluorescent Probes and Research Chemicals; 5th Edition 1992-94; Molecular Probes, Inc.

The particles will typically be at least about 1 weight %, usually at least 2 weight %, and up to 100 weight % of the non-volatile portion of the composition. Where the particles are dispersed in a vehicle, the weight % of the particles will generally be in the range of about 1-75 weight %, more usually about 1-50 weight %. The minor ingredients except for the medium will generally range from about 0.01 weight % to about 10 weight %, the amount generally being conventional for the purpose of the ingredient. Where the particles are sprayed as an aerosol, generally the particles will be present in the range of about 1 to 99 weight % of the composition.

Depending upon the need and the nature of the composition, the composition may be sprayed, wiped, smeared, painted, transferred from a template onto or proximal to the wound or may be made into a patch where the composition will be separate from or part of the adhesive. Alternatively, topically the composition may be applied to the wound and a dressing or other protective layer added to prevent contamination and abrasion. In some situations, the composition may be injected or dispensed from a tube, for example, during laparoscopic surgery, particularly where a minimally invasive surgical technique is employed and the rate of transdermal transport is insufficient to provide the pain relief required. Not more than one application will typically be required per 6 hours, usually per half-day, and times between applications may vary from 6 hours to 7 days, usually 12 hours to 4 days, where frequently by 7 days further treatment will not be required. During this time a therapeutically effective amount of the caine will be released from the particles.

The amount of the anesthetic salt applied to the wound area will be a therapeutically effective amount to minimize pain to a level that the patient can tolerate and preferably substantially eliminate any sense of pain. The amount of pain will usually vary with time, so that the amount of anesthetic that will be required can be diminished over time. Therefore, the profile of anesthetic release from the salt can be a diminishing amount of anesthetic being released over time. Conveniently, there may be an initial large release, less than about 30%, usually less than about 25%, of the total amount of anesthetic followed by a decreasing release over time at a lower amount at a therapeutic level. The large initial release coincides with the high levels of pain in the early post-operative period. After the initial release, generally not more than 60 weight %, more usually not more than about 50 weight %, will be released in 24 hours, where the pain alleviation is to occur over generally greater than two days, with diminishing percentages as the time for relief is extended.

The invention also provides a composition comprising a polymeric film having embedded therein drug salt particles of the invention. Such compositions can be used, for example, to deliver the drug salt particles to a tissue or anatomical site of a subject in need of treatment with the drug. For example, when the drug salt is a caine salt, the polymeric film composition can be applied to a wound bed. The drug particles are preferably substantially uniformly distributed through the film. In certain embodiments, the polymeric film is water soluble. In certain embodiments, the polymeric film has a melting point at or below physiological temperature, i.e., 37° C. In certain embodiments, the polymeric film is bioerodible or bioresorbable.

Suitable polymers for fabrication of the polymeric films of the invention include polyethylene glycol (PEG) of various molecular weights up to about 20,000, which would be expected to quickly dissolve under physiological conditions. Lower molecular weight PEG can also be used, including PEG with a molecular weight of 1000, which has a melting point of 34 to 36° C. Suitable polymers also include, but are not limited to, other water soluble polymers, such as homopolymers and copolymers, with molecular weights below 20,000, for example cellulose ethers, such as hydroxyethyl cellulose and hydroxypropyl cellulose; polyvinyl pyrrolidone; PEGylated polymers; polyvinyl alcohol; polyacrylamide; N-(2-hydroxypropyl)methacrylamide; divinyl ether-maleic anhydride; polyoxazoline; polyphosphates, polyphosphazenes; xanthan gum; pectins; chitosan derivatives, including N-acetyl chitosan; dextrans; carrageenans; guar gum; hyaluronic acid; albumin; starch and starch derivatives. The polymeric film can be composed of a single polymer or a combination of two or more polymers. In certain embodiments, the polymeric film is composed of a polymer blend.

In certain embodiments, the polymeric film is formed of multiple molecular weights of same polymer selected to provide desired chemical and/or physical properties. In certain embodiments, the polymeric film includes the polymer or polymers and a low molecular weight material for wetting of the drug particles which is combined with the polymer or polymers to enhance the mechanical properties of the film. For example, in certain embodiments the polymeric film includes PEG200 as a wetting agent, combined with PEG having a molecular weight of about 1,000 to 20,000. In certain embodiments, the particles are pre-treated with the wetting agent, such as PEG200, prior to embedding the particles in the polymeric film.

The polymeric film serves as a vehicle for administration of the drug to an anatomic site, for example, a biological surface, such as a wound bed, preferably resulting in a substantially uniform distribution of the drug particles to the biological surface. Preferably, the polymeric film melts, dissolves and/or degrades rapidly following administration to a subject and does not affect the uptake of the drug by the subject.

In one embodiment, a drug salt, such as a caine salt, of the invention is incorporated into rate controlling delivery tubes for the purposes of sustained release of the drug. These tubes can be applied to the tissue directly or incorporated into dressings, bandages, creams, ointments, gels and lotions to provide for the extended release of an agent, such as anesthetic agent, preferably a caine, over many days. The rate of drug release is determined by the diameter of the tubes containing the drug salt and the inherent solubility of the salt itself. The duration of drug release is determined by the length of the tube.

A tube of a defined diameter is chosen for the release flux and duration for a specific indication. The rate of delivery of the drug from the tube is proportional to the surface area of the open face or faces of the tube and the inherent solubility of the drug salt. In general the rate of dissolution is dependent upon the surface area to volume ratio of any substance. For example, a spherically shaped object from which dissolution takes place from the entire surface will show a progressively decreasing rate of release as the sphere shrinks in size and the surface area is reduced. Similarly a rod shaped solid drug salt particle will show a decrease in the rate of release characteristic of its geometric shape and the surface area to volume ratio. Limiting the dissolution to the surface of a three dimensional object, such as the open end of the tube, will only allow dissolution in 2 dimensions. The release from such a surface-only shape will therefore be constant with time. This is characterized as a zero order release and may be desirable for some drug delivery applications.

Other geometric shapes may also be employed to control the release kinetics of the anesthetic agent. Other shapes such as cubes, rectangles, cones, prisms, tetrahedrons, octahedron or any other shapes as may be readily derived may also be used in place of the aforementioned tube. Other shapes with open faces will provide other release kinetics as may be calculated by those skilled in the art providing a unique therapeutic release profile.

Although the discussion for the rate controlled delivery of a drug has been for tubes, any geometric shape may be employed for use in this invention. As examples one may employ a sphere with a hole, a cone with the base face exposed, a cube or rectangle with a face exposed. These and many other geometric shapes may be employed and all will provide a unique drug delivery profile dependent on the shape of drug containing object, the surface area exposed and the solubility of the drug salt employed. The delivery from such objects is readily calculated by those skilled in the art and can provide unique delivery profiles that may be desirable for certain applications.

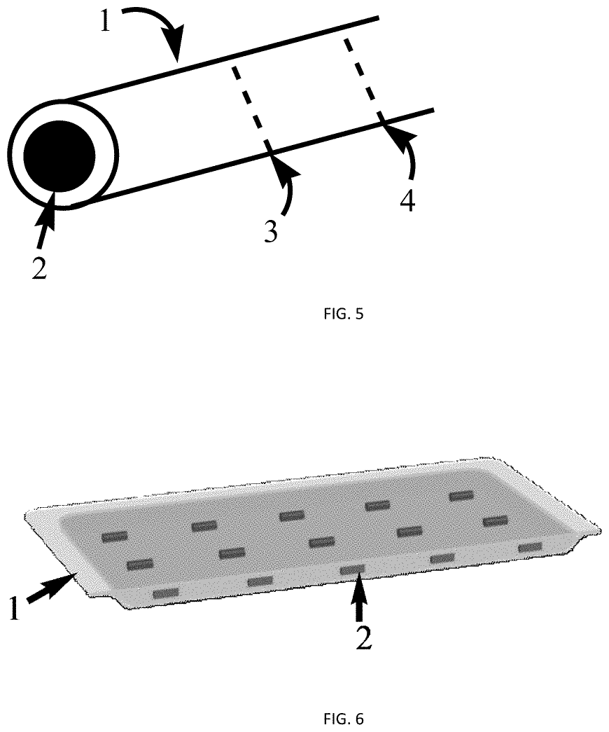

In one embodiment, the drug salt is encapsulated in an insoluble tube allowing for the exposure of the end faces of the tube to an aqueous environment allowing for the dissolution of the drug contained within. The tube can be cut to a specified length to provide a desired drug dose. This type of configuration is shown in FIG. 5, which shows open-ended tube 1, drug salt 2 incorporated in the interior of the tube and optional tube truncation points 3 and 4. Cutting the tube at either position 3 or 4 will provide different drug doses, with a cut at position 4 providing a higher dose than a cut at position 3. In either case, cutting the tube preferably produces a second open end in the resulting shortened tube.

In such a configuration dissolution of the drug will only take place on each cut end or face. As dissolution of the drug continues the drug will continue to erode down the tube continuously exposing new drug to the aqueous environment and providing a zero order release of the drug.

A larger diameter tube of drug will allow for a greater amount of drug delivered per unit time as the dissolution rate will be determined by the exposed surface area. The invention therefore allows for a wide range of drug delivery rates that depend upon the diameter of the tube used. Applications that require a small amount of drug to be delivered per unit of time will employ small diameter tubes. Applications requiring larger amounts of drug will use larger diameter tubes. This can be mathematically determined in advance knowing the drug dissolution rate per unit of exposed surface and by calculation knowing the desired drug concentration one may readily determine the amount of tubes of specified diameter to be used in the application.

The duration of release is controlled through the length of the tubes of drug employed. Longer tubes result in longer duration of release. Using both the tube diameter and the tube length allows one to design a drug release profile for any given amount of drug for any duration. The selection of tube diameter and tube length allows for the facile design of products that will last from hours to weeks and which can be readily calculated once one knows the dissolution rate of the drug in terms of mass released per unit time and unit area.

The use of an insoluble tube is not necessary if a relatively non-permeable coating is employed to provide a similar effect as a tube. The concept of a tube is used to describe a material which will allow little water or drug diffusion while retaining the drug in a reservoir. Many materials and designs can be envisioned as meeting these criteria. The tube may actually be a physical tube which is filled with a drug and is made of a thermoplastic materials such as polyethylene, polypropylene, nylon, polyester, urethane and generally of any material know to those skilled in the art that will maintain its structural properties while allowing for little diffusion of water into the tube or drug out of the tube. The tube is not a part of the delivery kinetics other than to act as a reservoir for remaining drug and allow the drug to dissolve from each exposed end surface of the tube.

The tube may also be made from a bioresorbable polymer meeting the aforementioned characteristics. A bioresorbable material would be one in which the tube material decomposes or degrades after the drug has eluted from the device. Such a material provides the benefit where it would be desirable to have no physically remaining tube after some period of time. One such example would be the use in a wound where the tubes may become incorporated into the wound with healing. Bioresorbable polymers such as polyesters, polyamides, polycarbonates and other materials known to those skilled in the art can be employed. The polymer may erode or absorb though either a bulk or surface degradation mechanism so long as it remains mostly intact for the duration of the drug delivery.

Additionally the tube may be prepared from thermoset materials if a particular longevity of the drug tubes is desired or if manufacturing of the drug product using such thermosets provides a design advantage. Any thermoset providing the aforementioned tube characteristics would be suitable such as epoxies, polyesters, polyurethanes and other polymeric materials that would be known to those skilled in the art.

Additionally the tube may be made from a bioresorbable inorganic material such as hydroxyapatite or combinations of an inorganic material and an organic polymer or inorganic polymer such as silicone to provide flexibility. The inorganic material may also be combined with bioresorbable organic polymers as described previously. Such a system may find use for bone surgery where the caine anesthetic would be part of the repair materials. Other materials known to those skilled in the art may also be employed in a similar manner.

The drug filled tubes used in the fabrication of a device may be prepared by a variety of techniques. Tubes may be filled using a molten form of the drug by injection filling or other means to introduce the molten drug into the tube. Once filled the drug filled tubes can be cut to length. Alternatively drug may be coextruded with a suitable plastic allowing for the simultaneous formation of drug filled tubing. This tubing may be subsequently cut to the appropriate length either during the formation of the drug filled tube or after the tubing has been prepared. Alternatively a molten form or a cooled tube wire form of the drug may be spray coated with an appropriate solution of a polymer meeting the described characteristics. This method allows for thin tube construction. Alternatively a drug extrusion may be coated by dipping or otherwise passing the molten drug through an appropriate molten polymer or solution of a polymer.

The drug containing tubes are incorporated into a device or into a topical or surgical product and become activated when wet. As one example the drug tubes can be added to a topical dressing or bandage to provide continuous release of an anesthetic caine drug. This is shown by example in FIG. 6 where the drug tubes are uniformly dispersed in the dressing material.

When the dressing is wetted, the dissolution of the drug begins from each tube and the drug diffuses throughout the dressing and into the contacting tissues. As long as the dressing remains wet, the drug will continuously be delivered to contacting tissue.

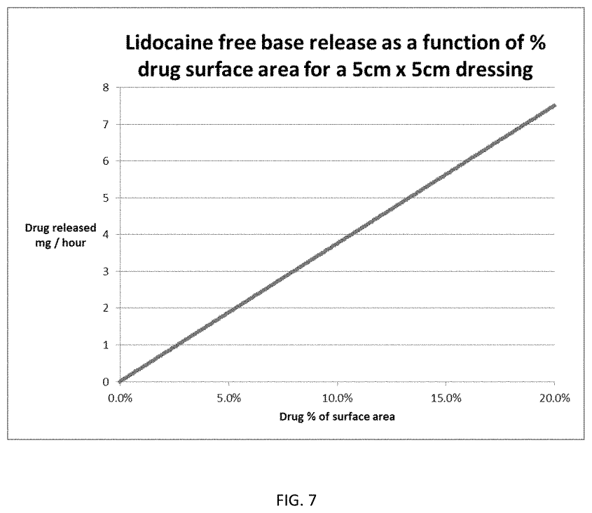

An example of the calculated delivery of the caine anesthetic from such a dressing is shown in FIG. 7. Based upon the diameter of the tube or the number of tubes used in a dressing and the solubility of the caine salt used the release rate is shown as a function of the surface area of the tube ends, that is of the total cross sectional area of both ends of the tube. This calculation assumes the drug has a dissolution constant of 1,500 micrograms per square centimeter per hour which is representative of the drug dissolution rates that can be achieved with a caine salt. The dressing size used for this calculation is 5 cm by 5 cm.

This example shows the wide range of drug delivery that is achievable with this invention showing the relationship between the cumulative surface area of exposed drug tubes and the area of the dressing or bandage.

The anesthetic tubes may also be employed in topical formulations in a variety of physical forms. For example, pastes, creams, lotions, gels, and liquids are all contemplated by the present invention. A difference between these forms is their physical appearance and viscosity, which can be governed by the presence and amount of emulsifiers and viscosity adjusters present in the formulation. Particular topical formulations can often be prepared in a variety of these forms. Solids are generally firm and will usually be in particulate form; solids optionally can contain liquids, emulsifiers, moisturizers, emollients, fragrances, dyes/colorants, preservatives and other active ingredients that increase or enhance the efficacy of the final product. Creams and lotions are often similar to one another, differing mainly in their viscosity; both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams may also contain liquids, emulsifiers, moisturizers, emollients, fragrances, dyes/colorants, preservatives and other ingredients that increase or enhance the efficacy of the final product. Liquids are thinner than creams, lotions, or gels and often do not contain emulsifiers.

Applications include such examples as thermal burns, sun burns, friction burns, hemorrhoids, abrasions, lacerations, dermal penetrations and any similar injury where the treatment of pain is desired. The anesthetic agent may be combined with other active medicaments in such products such as antibiotics, antibacterials, sun screens or other ingredients that are used for the intended use of the product.

In such topical applications the anesthetic tubes are added during the application of the topical product to activate and initiate the release of the anesthetic agent. This may be accomplished in a variety of ways that allow the mixing of the drug eluting tubes into the composition. For example the tubes may be contained in a separate compartment of a two part dispenser. A membrane separating the two components is broken by finger pressure allowing the mixing of the two components which are subsequently mixed by kneading the packaging. The product is subsequently dispensed for the intended application. In another delivery method the anesthetic tubes are contained in a nonaqueous vehicle such as propylene glycol where the solubility of the caine salt is low. This liquid is contained in a two part tube and mixing of the aqueous lotion or cream is accomplished when product is squeezed from the container. Alternatively the anesthetic tubes are simply mixed with the product prior to administration. There are many means by which the free flowing anesthetic tubes may be combined with a topical product by those skilled in the art to achieve the activation of the anesthetic tubes and the release of the caine anesthetic.

In dressing or bandage applications the anesthetic caine tubes are integral to the manufacture of the product. The product is stored in a dry state and activated at time of use by wetting the dressing with moisture. Alternatively the dressing may be stored pre-wetted with a nonaqueous agent such as propylene glycol. Application of this dressing to a wound will result in the absorption of water which will initiate the release of the caine anesthetic.

Once the particles have been prepared, irrespective of the method employed in their preparation, the particles are sized and fractioned typically by sieving operations, although other methods may be employed. To control particle distribution and particle size a typical sieving operation would employ at least 2 sieves of the appropriate size. The larger sieve size would allow for the rejection of particles larger than the specified maximum while the lower sieve size would serve to retain the particles of the specified size. The selection of the sieves determines the particle size distribution. Using this approach one can also prepare multimodal distributions to obtain different release profiles of drug. Nominal particle size and particle size distribution is determined by an instrument such as a Coulter LS13 on suspensions of the microparticles.

Drug dissolution kinetics are evaluated using an LC method employing an infinite sink concept. A known amount of microparticles are suspended in a defined volume of a suitable test medium, for example a phosphate buffer solution containing 1% Tween 80, meant to simulate in vivo release kinetics. The suspension of microparticles is kept at a constant temperature, typically 37° C., for a period of time, for example, about 12 hours, with constant agitation. The particles are removed from the solution by filtration and re-suspended in another fresh amount of the test media. The original solution is assayed for the amount of drug product in solution by an appropriate quantitative method, typically an LC method employing UV detection or MS.

If fluorescent or colored microparticles are desired the procedure for making the microparticle is followed, however, for a fluorescent product a compound such as fluorescein is added to the mixture before the precipitation or preparation of the microparticle is attempted. If a colored product is required a food safe dye such as FD&C Blue No 1 or Blue No 2 is used.

Drug product of the appropriate size is combined with other agents that may be appropriate to provide free flowing stable microparticles and added to an appropriate aerosol container. The aerosol container is subsequently pressurized with a high purity propellant and sealed under pressure with the appropriate spray nozzle to provide the spray pattern desired and in some cases to provide a metered dose of the drug. Alternatively, the drug product can be suspended into a PBS solution or other suitable vehicle just prior to application to the wound. The product is distributed over the wound by spraying using a variety of possible propulsion systems e.g. an air brush type of system, pump sprayer system, etc., whereby drug product suspended in the PBS is aspirated through a tube using the Venturi concept with a propellant container.

The acid addition salts of the invention can be prepared by methods known in the art. For example, an acid addition salt of a basic drug in accordance with the invention may be prepared by any conventional means, including precipitation of the salt from solution, spray drying a solution of the salt, reaction of the drug and acid in solution and removal of solvent, or fusion of the free base of the drug with the acid. In one embodiment the free base of the drug compound is combined with the acid in a suitable solvent, such as water or a polar organic solvent. Alternatively, a salt of the drug, such as the hydrochloride salt, is reacted with a salt of the acid, for example, the sodium salt, in water or a polar organic solvent. In either case, the desired salt can either spontaneously precipitate upon formation or be induced to precipitate by adding a suitable cosolvent and/or concentrating the solution. In certain embodiments, the free base of the drug is combined with the acid in the absence of solvent, resulting in the formation of the desired salt.

### EXAMPLES

### Example 1 Preparation of Bupivacaine 4-Bromobenzenesulfonate Salt

To a solution of 4-bromo-benzenesulfonic acid (2.55 g, 10.0 mmol) in ether (50 mL) and acetone (5 mL) at room temperature was added a solution of bupivacaine (2.95 g, 10.2 mmol) in ether (20 mL) and acetone (5 ml). The reaction mixture stirred overnight, the solid was filtered and washed with ether (2×), and dried in vacuo to give the desired salt (4.3 g, 82%) as a white solid. 1H NMR (300 MHz, CD₃OD) δ 7.72-7.69 (d, J=7.8 Hz, 2H), 7.58-7.56 (d, J=8.1 Hz, 2H), 7.16-7.10 (m, 3H), 4.10-4.06 (d, J=10.2 Hz, 1H), 3.70-3.65 (d, J=12.6 Hz, 1H), 3.17-3.07 (t, J=8.4 Hz, 3H), 2.42-2.36 (d, J=11.7 Hz, 1H), 2.21 (s, 7H), 1.99-1.71 (m, 8H), 1.46-1.33 (m, 2H), 1.00-0.95 (t, J=7.5 Hz, 3H).

### Example 2 Preparation of Bupivacaine 3,4-Dibromobenzenesulfonate Salt

### (a) 3,4-Dibromobenzenesulfonic acid

A mixture of 3,4-dibromobenzene sulfonyl chloride (4.90 g, 14.6 mmol) in dioxane/water (20/10 mL) was heated to 100° C. for 6 h. The reaction was cooled to room temperature and lyophilized to afford 3,4-dibromobenzenesulfonic acid the title compound (4.68 g, 99%). LC-MS: 314.7 [M-H]⁻; 1H NMR (300 MHz, DMSO-d6) δ 7.81 (s, 1H), 7.75 (d, J=7.8 Hz, 1H), 7.43 (d, J=7.8 Hz, 1H).

### (b) Bupivacaine 3,4-Dibromobenzenesulfonate Salt

A solution of 3,4-dibromobenzenesulfonic acid (1.6 g, 5.06 mmol) and bupivacaine (1.6 g, 5.55 mmol) in acetone (5 mL) and ether (40 mL) was stirred overnight. The oil at the bottom of the reaction vessel solidified slowly. The solvent was removed, and the solid was triturated with ether (2×) and dried to yield bupivacaine 3,4-dibromobenzenesulfonate salt (2.47 g, 81%) as a white solid. ¹H NMR (300 MHz, CD₃OD) δ 8.06-8.05 (d, J=1.8 Hz, 1H), 7.75-7.72 (d, J=8.4 Hz, 1H), 7.66-7.62 (dd, J=1.8, 2.1 Hz, 1H), 7.17-7.09 (m, 3H), 4.10-4.06 (d, J=11.7 Hz, 1H), 3.70-3.66 (d, J=12.6 Hz, 1H), 3.17-3.07 (t, J=8.4 Hz, 1H), 2.42-2.36 (d, J=9.3 Hz, 1H), 2.21 (s, 6H), 1.99-1.46 (m, 9H), 1.38-1.33 (m, 2H), 1.00-0.95 (t, J=7.5 Hz, 3H).

### Example 3 Preparation of Bupivacaine 2,4-Dibromo-3-methylbenzenesulfonate Salt

### (a) 2,4-Dibromo-3-methylbenzenesulfonic acid

To a solution of 2,6-dibromotoluene (10.0 g, 40 mmol) in 1,2-dichloroethane (40 mL) chlorosulfonic acid (4.66 g, 40 mmol, 1 eq) was added dropwise. The reaction mixture was stirred at room temperature for 3 days, and monitored by TLC and LC-MS. After concentrated to about 20 mL, solid precipitated out. The solid was filtered and washed with hexane, and dried in vacuo to yield 2,4-dibromo-3-methylbenzenesulfonic acid (3.65 g, 28%) as pale white solid. LC-MS: 328.7 [M-H]⁻; ¹H NMR (300 MHz, CD₃OD): δ 7.83 (d, J=9.0 Hz, 1H), 7.62 (d, J=9.0 Hz, 1H), 2.66 (s, 3H).

### (b) Bupivacaine 2,4-Dibromo-3-methylbenzenesulfonate Salt

A solution of 2,4-dibromo-3-methylbenzenesulfonic acid (2.29 g, 6.94 mmol) and bupivacaine (2.5 g, 8.68 mmol) in acetone (5 mL) and ether (40 mL) was stirred overnight. Solid formed was filtered and washed with ether, dried in vacuo to afford bupivacaine 2,4-dibromo-3-methylbenzenesulfonate salt (2.55 g, 59%) as pale white solid. ¹H NMR (300 MHz, CD₃OD): δ 7.83-7.80 (d, J=7.5 Hz, 1H), 7.61-7.58 (d, J=8.1 Hz, 1H), 7.18-7.09 (m, 3H), 4.18-4.12 (dd, J=3.6, 4.2 Hz, 1H), 3.70-3.65 (d, J=12.9 Hz, 1H), 3.19-3.10 (m, 3H), 2.64-2.63 (m, 2H), 2.42-2.38 (d, J=11.1 Hz, 1H), 2.20 (s, 7H), 1.99-1.69 (m, 9H), 1.45-1.33 (m, 2H), 1.00-0.95 (t, J=7.5 Hz, 3H).

### Example 4 Preparation of Lidocaine 4-Tetradecylbenzenesulfonate Salt

### (a) 4-Tetradecylbenzenesulfonic acid

To a solution of tetradecylbenzene (10.0 g, 36.4 mmol) in 1,2-dichloroethane (40 mL) was added dropwise a solution of chlorosulfonic acid (4.24 g, 36.4 mmol) in 1,2-dichloroethane (10 mL). The deep brown solution was stirred at room temperature while solid started to precipitate after 30 minutes. After stirring overnight, the solvent was removed to give a brownish crude product. Recrystallization in toluene (˜50 mL) afforded 4-tetradecylbenzenesulfonic acid (9.6 g, 76%) as a white solid. ¹H NMR (300 MHz, CDCl₃) δ 8.31 (brs, 3 H), 7.74 (d, J=7.2 Hz, 2H), 7.20 (d, J=7.2 Hz, 2H), 2.61 (t, J=7.5 Hz, 2H), 1.57 (m, 2H), 1.25 (m, 22H), 0.88 (t, J=7.5 Hz, 3H).

### (b) Lidocaine 4-tetradecylbenzenesulfonate salt

A mixture of 4-tetradecylbenzenesulfonic acid (4.0 g, 11.3 mmol) and lidocaine (2.93 g, 12.5 mmol) in isopropanol (40 mL) was heated to form a clear solution. The solution was cooled to room temperature, and stood overnight. The solid was filtered and washed with isopropanol (10 mL), and ether (2×20 mL), and dried to give lidocaine 4-tetradecylbenzenesulfonate salt (6.0 g, 90%) as white solid. ¹H NMR (300 MHz, CD₃OD) δ 7.73-7.70 (d, J=8.1 Hz, 2H), 7.24-7.22 (d, J=7.8 Hz, 2H), 7.12 (m, 3H), 4.89-4.87 (m, 2H), 4.22 (s, 2H), 3.36-3.34 (m, 3H), 2.67-2.61 (t, J=7.8 Hz, 2H), 2.23 (s, 4H), 1.61 (m, 2H), 1.40-1.35 (t, J=7.5 Hz, 6H), 1.32-1.23 (m, 16H), 0.92-0.87 (m, 6H).

### Example 5 Preparation of Lidocaine 4-Octadecylbenzenesulfonate Salt

### (a) 4-Octadecylbenzenesulfonic acid

To a solution of n-octadecylbenzene (5.0 g, 15.1 mmol) in 1,2-dichloroethane (20 mL) was added dropwise a solution of chlorosulfonic acid (1.76 g, 15.1 mmol) in 1,2-dichloroethane (10 mL). The resulting deep brown solution was stirred at room temperature while solid started to precipitate out after 30 minutes. After stirring overnight, the solvent was removed in vacuum to give a brownish crude product. Recrystallization of the solid in toluene (˜50 mL) afforded 4-octadecylbenzenesulfonic acid (3.0 g, 48%) as a light grey solid. LC-MS: 408.9 [M-H]⁻ ¹H NMR (300 MHz, CD₃OD) δ7.72 (d, J=8.4 Hz, 2H), 7.18 (d, J=8.4 Hz, 2H), 2.59 (t, J=7.5 Hz, 2H), 1.57 (m, 2H), 1.25 (m, 30H), 0.88 (t, J=7.5 Hz, 3H).

### (b) Lidocaine 4-Octadecylbenzenesulfonate Salt

A mixture of 4-octadecylbenzenesulfonic acid (4.0 g, 9.7 mmol) and lidocaine (2.5 g, 10.7 mmol) in ethanol (50 mL) and water (25 mL) was heated to form a clear solution. The solution was cooled to room temperature, and stood overnight. The solid was filtered and washed with isopropanol (2×10 mL), and dried for three days to give lidocaine 4-octadecylbenzenesulfonate salt (5.5 g, 87%) as a white solid. ¹H NMR (300 MHz, CD₃OD) δ 7.73-7.70 (d, J=8.4 Hz, 2H), 7.24-7.22 (d, J=8.1 Hz, 2H), 7.14-7.10 (m, 3H), 4.89-4.87 (m, 2H), 4.22 (s, 2H), 3.36-3.34 (m, 3H), 2.66-2.61 (t, J=7.5 Hz, 2H), 2.23 (s, 4H), 1.61 (m, 2H), 1.40-1.35 (t, J=7.5 Hz, 6H), 1.32-1.23 (m, 24H), 0.92-0.87 (m, 6H).

### Example 6 Preparation of Lidocaine 4-Docosanylbenzenesulfonate Salt

### (a) 4-Docosanylbenzenesulfonic acid

To a solution of docosanylbenzene (11.5 g, 30.0 mmol) in 1,2-dichloroethane (80 mL) was added dropwise a solution of chlorosulfonic acid (3.51 g, 30 mmol) in 1,2-dichloroethane (20 mL). After stirring overnight, the solid was filtered and washed with hexane (3×), and recrystallized from toluene, and dried to give 4-docosanylbenzenesulfonic acid (5.1 g, 36%) as a light grey solid. ¹H NMR (300 MHz, CD₃OD) δ 10.37 (brs, 3H) 7.82 (d, J=8.4 Hz, 2H), 7.34 (d, J=8.1 Hz, 2H), 2.68 (t, J=7.8 Hz, 2H), 1.61 (m, 2H), 1.40-1.10 (m, 33H), 0.88 (t, J=6.9 Hz, 3H).

### (b) Lidocaine 4-Docosanylbenzenesulfonate Salt

A mixture of 4-docosanylbenzenesulfonic acid (3.0 g, 6.4 mmol) and lidocaine (1.66 g, 7.1 mmol) in ethanol (30 mL) was heated to form a clear solution. The solution was cooled to room temperature, and stood overnight. The solid was filtered and washed with isopropanol (2×15 mL) and dried to give lidocaine 4-docosanylbenzenesulfonate salt (3.2 g, 71%) as a light grey solid. ¹H NMR (300 MHz, CD₃OD) δ 7.73-7.70 (m, 2H), 7.24-7.10 (m, 5H), 4.89-4.87 (m, 2H), 4.22 (s, 2H), 3.36-3.34 (m, 3H), 2.66-2.61 (m, 2H), 2.23 (s, 4H), 1.61 (m, 2H), 1.40-1.23 (m, 38H), 0.92-0.87 (m, 6H).

### Example 7 Preparation of Bupivacaine 3,5-Dibromo-4-methylbenzenesulfonate Salt

### (a) 3,5-Dibromo-4-methylbenzene sulfonic acid

To a solution of 3,5-dibromo-4-methylbenzenamine (43 g, 162 mmol, 1.0 eq) in a mixture of water (430 mL) and conc. HCl (43 mL) at 0° C. was added a solution of NaNO₂ (13.4 g, 195 mmol, 1.2 eq) in water (100 mL) slowly. Then the mixture was stirred for 20 min at 0° C. to form a diazonium solution. This diazonbium solution was added to a solution of CuCl (1.61 g, 16.2 mmol, 0.1 eq), CuCl2 (6.52 g, 48.6 mmol, 0.3 eq) and sulfur dioxide (3 M in acetic acid, 516 mL) in toluene (516 mL) at room temperature. The mixture was then heated to 45° C. and stirred for 1 h. The mixture was then cooled to room temperature. The organic and aqueous phases were separated, and the aqueous layer was extracted with ethyl acetate (2×300 mL). The organic phase was dried over sodium sulfate and concentrated. The residue was purified by flash column chromatography to yield 3,5-Dibromo-4-methylbenzene-1-sulfonyl chloride (22 g, 39%). TLC:petroleum ether:ethylacetate=1:0, Rf (2)=0.5

A solution of 3,5-dibromo-4-methylbenzene-1-sulfonyl chloride (20 g, 57.3 mmol) in water (40 mL) and 1,4-dioxane (80 mL) was heated to 100° C. and stirred for 1 h. TLC showed the reaction was complete and the mixture was concentrated. The residue was lyophilized overnight. The solid was triturated with petroleum ether several times, and dried to give 3,5-dibromo-4-methylbenzenesulfonic acid (14.1 g, 74%) as a light pink solid. ¹H NMR (400 MHz, CD₃OD) δ 7.94 (s, 2H), 2.58 (s, 3H).

### (b) Bupivacaine 3,5-Dibromo-4-methylbenzenesulfonate Salt

To a hot solution of 3,5-dibromo-4-methylbenzenesulfonic acid (6.6 g, 20 mmol) in acetone (60 mL) and methanol (10 mL) was added a hot solution of bupivacaine (6.05 g, 21 mmol, 1.05 eq) in acetone (10 mL). To the solution was added ether (20 mL). After 10 h at room temperature, the solid was filtered and washed with acetone (30 mL) and ether (20 mL) and dried in vacuo to provide bupivacaine 3,5-dibromo-4-methylbenzenesulfonate salt (9.88 g, 80%) as a white solid. ¹H NMR (400 MHz, CD₃OD) δ 7.93 (s, 2H), 7.12 (s, 3H), 4.10 (brd, J=1.2 Hz, 1H), 3.67 (brd, J=1.2 Hz, 1H), 3.15 (t, J=1.3 Hz, 4H), 2.57 (s, 3H), 2.40 (brd, 1H), 2.20 (s, 6H), 2.04-1.64 (m, 8H), 1.45-1.33 (m, 2H), 0.98 (t, J=7.2 Hz, 3H).

### (c) Particle Preparation

Particles of bupivacaine 3,5-dibromo-4-methylbenzenesulfonate salt, nominal size 650 nM, were by melting solid bupivacaine 3,5-dibromo-4-methylbenzenesulfonate salt (3-5 g) in a nitrogen atmosphere in a glass container immersed in oil bath preheated to a temperature 215° C. (approximately 10° C. above the melting temperature of the salt). After 5 minutes of thermal treatment the melted material was cooled to ambient temperature over 30 minutes under nitrogen. The resulting product was gently crushed using a mortar and pestle and sieved using a stainless-steel sieve set. The product fraction retained between the sieves with opening size 600 uM and 725 uM were isolated with an average 8% yield. The particles had a melting temperature of 196.2° C. and a specific melting enthalpy of 62.5 J/g as measured by differential scanning calorimetry (Pyrus 1 DSC, Perkin Elmer).

### Example 8 Preparation of Lidocaine 3,5-Dibromo-4-methylbenzenesulfonate Salt

To a hot solution of 3,5-dibromo-4-methylbenzene sulfonic acid (6.6 g, 20 mmol) in acetone (60 mL) and methanol (10 mL) was added a solution of lidocaine (4.92 g, 21 mmol, 1.05 eq) in acetone (10 mL). The crystals formed quickly. After sitting overnight at room temperature, the solid was filtered and washed with acetone (2×10 mL), and dried in vacuo to give lidocaine 3,5-dibromo-4-methylbenzene sulfonate (8.37 g, 74%) as white solid. ¹H NMR (400 MHz, CD₃OD) δ 7.93 (s, 2H), 7.11 (brs, 3H), 4.24 (s, 2H), 3.34 (q, J=7.2 Hz, 4H), 2.22 (s, 6H), 1.37 (t, J=7.2 Hz, 6H).

### Example 9 Preparation of Bupivacaine 4-Bromobenzenesulfonate Salt

To a solution of 4-bromobenzenesulfonic acid-hydrate (10 g, 39.2 mmol) in acetone (20 mL) and ether (200 mL) was added a solution of bupivacaine (11.54 g, 40.0 mmol, 1.02 eq) in acetone (50 mL). Solid was formed immediately. After standing overnight, the solid was filtered and washed with ether (50 mL), and dried. The solid was dissolved in hot isopropanol (250 mL), and cooled to rt and stood overnight. The crystals formed were filtered and washed with isopropanol (2×25 mL), and dried in vacuo to give bupivacaine 4-bromobenzenesulfonate salt (18.1 g, 61%) as a white solid.

¹H NMR (300 MHz, CD₃OD) δ 7.71 (d, J=8.5 Hz, 2H), 7.57 (d, J=8.5 Hz, 2H), 7.18-7.09 (m, 3H), 4.08 (brd, J=10.6 Hz, 1H), 3.68 (brd, J=10.6 Hz, 1H), 3.19-3.08 (m, 3H), 2.39 (br d, J=13.6 Hz, 1H), 2.21 (s, 6H), 2.20-1.63 (m, 7H), 1.46-1.33 (m, 2H), 0.98 (t, J=6.90 Hz, 3H).

### Example 10 Preparation of Lidocaine 4-Bromobenzenesulfonate Salt

To a solution of 4-bromobenzenesulfonic acid-hydrate (10 g, 39.2 mmol) in acetone (20 mL) and ether (200 mL) was added a solution of lidocaine (9.37 g, 40.0 mmol, 1.02 eq) in acetone (80 mL). After 30 min, an oil was formed, which turned into solid while stirring. After being stirred for 3 h, the solid was filtered and washed with ether (2×100 mL), and dried. The solid was dissolved in hot isopropanol (100 mL), and cooled to rt overnight. The crystals formed were filtered and washed with isopropanol (2×25 mL), and dried in vacuo to give lidocaine 4-bromobenzenesulfonate salt (11.2 g, 88%) as a white solid.

¹H NMR (300 MHz, CD₃OD) δ 7.71 (d, J=8.3 Hz, 2H), 7.57 (d, J=8.3 Hz, 2H), 7.18-7.09 (m, 3H), 4.24 (s, 2H), 3.34 (q, J=7.28 Hz, 4H), 2.22 (s, 6H), 1.38 (t, J=7.28 Hz, 6H).

### Example 11 Pharmacokinetics of Caine Anesthetic Salts in Female Sprague Dawley Rats after a Single Subcutaneous Dose

### Test Articles

1. Bupivacaine 4-bromobenzenesulfonate salt, nominal particle size 650 μm (B-4BrBSA-650)
2. Bupivacaine 3,5-dibromo-4-methybenzenesulfonate salt, nominal particle size 650 μm (B-35BrBSA-650)
3. Lidocaine 4-bromobenzenesulfonate salt, nominal particle size 650 μm (L-4BrBSA-650)
4. Lidocaine 3,5-dibromo-4-methybenzenesulfonate salt, nominal particle size 650 μm (L-35BrBSA-650)

This study was designed to evaluate the pharmacokinetic consequences of using particles suspended in liquid administered subcutaneously in the rat. The test articles are drug compositions consisting of B-4BrBSA-650, B-35BrBSA-650, L-4BrBSA-650, and L-35BrBSA-650 particles in a polyethylene glycol 200 vehicle (PEG200; Sigma-Aldrich, polyethylene glycol 200 Bioultra). The test articles were prepared by adding a volume of 100 of PEG200 to an appropriate weighed amount of test article in a vial, gently mixed, and then applied uniformly via a single subcutaneous dose via incision on the dorsum of female Sprague Dawley rats.

Methods: Twenty (20) female, jugular vein catheterized (JVC) CD rats were assigned to four dose groups consisting of five animals per group (Animals 1-5: B-4BrBSA-650; Animals 6-10: B-35BrBSA-650; Animals 11-15: L-4BrBSA-650, and Animals 16-20: L-35BrBSA-650). Doses were administered via an incision on the dorsal region, as a subcutaneous liquid application (suspension of powder). Each test article was administered at an amount determined by each animal's weight using the calculated mg per kg-bw (bodyweight) as shown in the table below.

| | | | Dose | | Dose |
|---|---|---|---|---|---|
| | Animal | | Level | Vehicle | Amount |
| Group | ID | Compound | (mg/kg-bw) | (Volume) | (mg) |
| 1 | 1 | B-4BrBSA-650 | 114 | 100 μL | 27.2 |
| | 2 | | | PEG200 | 27.8 |
| | 3 | | | | 26.2 |
| | 4 | | | | 26.7 |
| | 5 | | | | 26.8 |
| 2 | 6 | B-35BrBSA-650 | 134 | 100 μL | 33.4 |
| | 7 | | | PEG200 | 32.2 |
| | 8 | | | | 33.2 |
| | 9 | | | | 31.8 |
| | 10 | | | | 30.3 |
| 3 | 11 | L-4BrBSA-650 | 161 | 100 μL | 38.8 |
| | 12 | | | PEG200 | 39.8 |
| | 13 | | | | 37.2 |
| | 14 | | | | 39.6 |
| | 15 | | | | 38.5 |
| 4 | 16 | L-35BrBSA-650 | 193 | 100 μL | 48.3 |
| | 17 | | | PEG200 | 48.1 |
| | 18 | | | | 47.3 |
| | 19 | | | | 46.5 |
| | 20 | | | | 47.7 |

Test articles 1 to 4 were provided as a powder in individual vials. The vehicle was provided as a liquid in an individual container. On the day of testing, each animal's dose was individually weighed and placed into an appropriately sized vial. A volume of 100 μL of vehicle was added to each individual dose, allowed to soak, and shaken occasionally to uniformly mix the particles with the PEG200.

For each animal, an incision was made in the dorsum to the rear of the jugular vein cannula incision, and the test article carefully deposited into the subcutaneum. For all groups, the test article suspension was applied to the subcutaneum using a plastic spatula. The test article was distributed as evenly as possible. Incisions were closed with staples, and the animals allowed to recover. All animals received analgesia and antibiotic treatment.

Cage side observations were performed daily. Blood samples for pharmacokinetic (PK) analyses were collected at pre-dose and at 1, 4, 8, 24, 48, 72, and 96 hours post-dose, processed to plasma and stored frozen at −60 to −80° C. until transferred to the Analytical Department at Pacific BioLabs for analysis. At study termination, all animals were euthanized and the dose sites were exposed for gross evaluation of residual test articles and local response to the injected materials. The subcutaneous tissues surrounding the dose sites were collected, weighed, and stored frozen at −60 to −80° C. for possible analysis.

Results and Discussion: Approximately 1 hour following dose administration, all Group 3 animals had sudden onset ataxia, shallow respiration, slight hypothermia, and intermittent full body stiffness. All animals were placed under a warming lamp. Approximately 30 minutes following onset, Animal #14 appeared stiff with extended limbs and was unresponsive to stimuli, with very slow and shallow respiration. A volume of 5 mL of Lactated Ringer's Solution was administered subcutaneously. Approximately 45 minutes following onset, Animals #11, #12, #13, and #15 became alert and mobile, and were returned to caging. No changes in symptoms were apparent in Animal #14 until approximately 1.5 hours following onset, when some responsiveness and mobility returned. Within an additional 15 minutes, the animal appeared alert and normally mobile. No additional abnormal clinical symptoms were observed over the duration of the study.

All scheduled blood collections for pharmacokinetic analysis were collected within the specified range, with the exception of the blood sample collected from Group 4 Animal #19 at the 4-hour collection.

For Group 1 (test article 1; B-4BrBSA-650+PEG200), two out of five animals lost weight over the course of the study. Weight loss was less than 5% total body weight. This weight loss may not be considered abnormal or due to test article. Slight weight loss may be attributable to the surgical procedures and stress of multiple sample collection. On average, the animals gained 4 grams at the end of the study.

Dose site observations included very slight to slight vascularization surrounding the incision sites. Hematomas were present in Animals #2 and #4, with encapsulation of clotted blood in Animal #4. No additional encapsulation or residual test article was visible.

For Group 2 (test article 2; B-35BrBSA-650+PEG200), two out of five animals lost weight over the course of the study. Weight loss was less than 5% total body weight. This weight loss may not be considered abnormal or due to test article. Slight weight loss may be attributable to the surgical procedures and stress of multiple sample collection. On average, the animals gained 1 gram at the end of the study. Dose site observations included very slight to slight vascularization surrounding the incision sites. A small hematoma was present in Animal #7. Small areas of residual test article were visible at or near the incision site in all animals. No encapsulation was present.

For Group 3 (test article 3; L-4BrBSA-650+PEG200), four out of five animals lost weight over the course of the study, resulting in a group mean body weight loss (−1 gram at the end of the study). Weight loss was less than 5% total body weight except for Animal #14. The weight loss may be attributable to the stress of the apparent dose reaction following administration on Day 0. Dose site observations included moderate to severe vascularization surrounding the incision sites. Encapsulation from 0.5 cm to 3 cm was present in three animals. Areas of an unknown brown material were present in Animal #13. No residual test article was visible.

For Group 4 (test article 4; L-35BrBSA-650+PEG200), two out of five animals lost weight over the course of the study. Weight loss was less than 5% total body weight. The weight loss may not be considered abnormal or due to test article. Slight weight loss may be attributable to the surgical procedures and stress of multiple sample collection. On average, the animals gained 1 gram at the end of the study. Dose site observations included very slight to moderate vascularization surrounding the incision sites. No residual test article or encapsulation was visible.

Dose site weights ranged from approximately 2.3 to 5.1 grams in Group 1, 2.6 to 4.7 grams in Group 2, 3.1 to 6.1 grams in Group 3, and 2.0 to 2.7 grams in Group 4. Dose site weights were dependent on the total area of skin collected, determined by the general location of the incision and reaction, and extent of visible reaction or residual test article.

One animal from each group was selected for a general gross necropsy. No abnormalities in major organs were apparent in any of the examined animals.

In summary, the results indicate that test articles B-4BrBSA-650, B-35BrBSA-650, and L-35BrBSA-650 were well tolerated as administered. Test article L-4BrBSA-650 resulted in abnormal clinical symptoms, including unresponsiveness, ataxia, and weight loss following dose administration, but all animals survived until scheduled euthanasia. There does appear to be a difference in the overall tissue response to the test articles as administered.

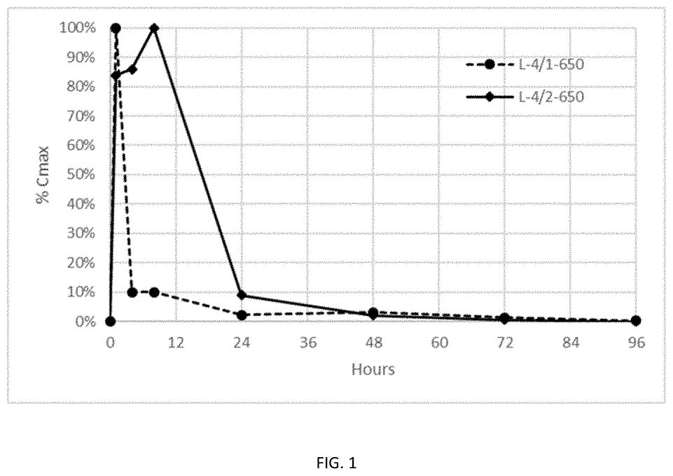

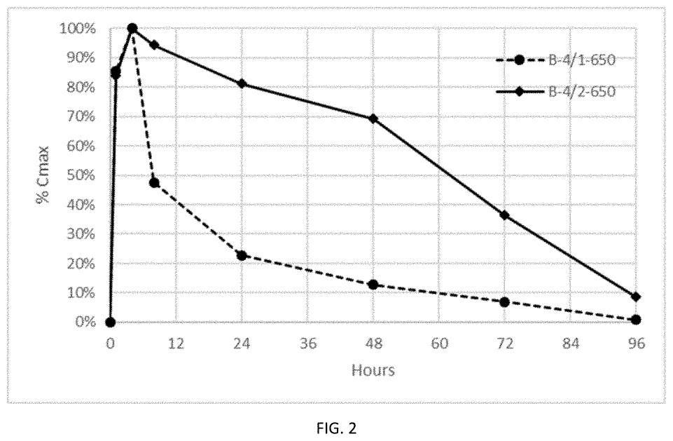

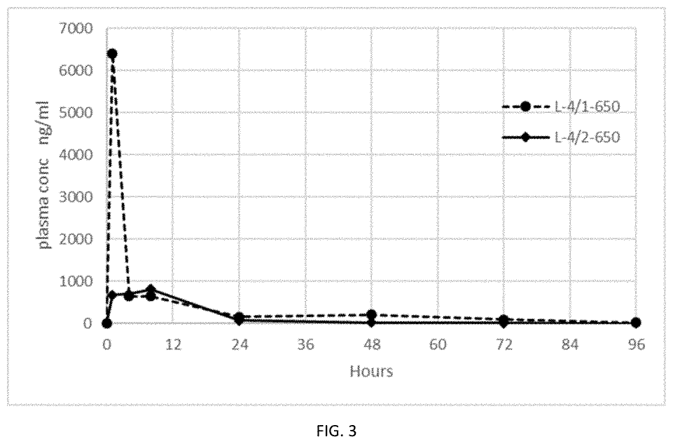

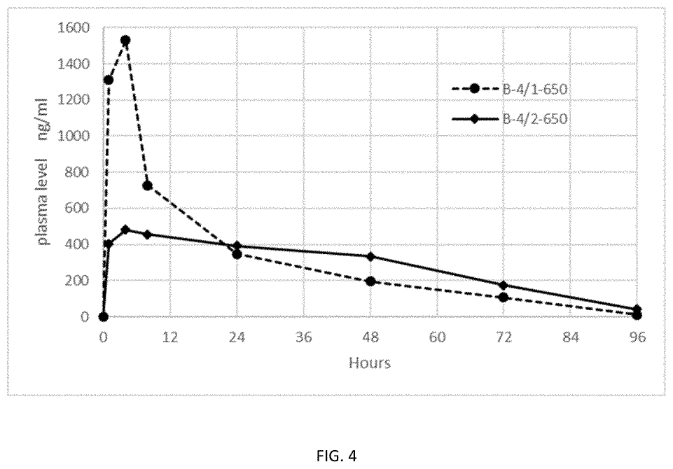

The results of the pharmacokinetic analysis are presented in FIGS. 1-4. FIG. 1 is a graph comparing the average plasma bupivacaine concentration for Groups 1 and 2 normalized to the bupivacaine Cmax of the specified salt versus time. FIG. 2 presents the same data as plasma bupivacaine concentration (ng/mL) versus time. The results show that test article 2 provides a greater duration of delivery of bupivacaine compared to test article 1 and without the initial spike in bupivacaine plasma concentration exhibited by test article 1. FIG. 3 is a graph of average plasma lidocaine concentration for groups 3 and 4 normalized to the lidocaine Cmax of the specified salt versus time. FIG. 4 presents the same data as plasma lidocaine concentration (ng/mL) versus time. The results show that test articles 3 and 4 provide delivery of lidocaine over at least 24 hours, while test article 4 avoids the initial spike in plasma lidocaine concentration exhibited by test article 3.

### Example 12 Preparation of Model Polymeric Films Comprising Particles

To demonstrate the ability of PEG1000 to form particle containing films, PEG1000/disodium hydrogen phosphate decahydrate Na₂HPO₄.10H₂O compositions containing 20% wt/wt and 30% wt/wt of sodium phosphate particles with size 100-250 microns were prepared. PEG1000 (20 g) was placed in a 50-ml glass container and melted in a water bath preheated to 50° C. To prepare 20% and 30% particle containing PEG1000 compositions, melted PEG1000 (5 g) was combined with the appropriate amount of phosphate (see Table 1). The compositions were mixed thoroughly by spatula, and composition temperature was maintained at 50° C. before film forming.

Particle containing PEG1000 films, approximately 1″×4″ in size, were formed by dispersing the liquid PEG1000 compositions on the surface of polyethylene film (PE film, thickness—2 mils) with a flat stainless steel bar. The thickness of the PEG1000 films was maintained by using two spacers (thickness 15 mils or 20 mils) supporting flat bar. The temperature of PEG1000 composition was brought to ambient and the surface of the solidified films was covered doubled with a protective layer of 2 mil thick PE film. The film was easily detached from the PE protective film. The estimated PEG1000 film phosphate particle content (mg/square inch) is reported in the table below.

PEG1000 films containing disodium hydrogen phosphate decahydrate particles

| | PEG1000 | Phosphate particles | | Spacer | Resulting film | Solid content, |
|---|---|---|---|---|---|---|
| Sample | amount, g | size, um | amount, g | thickness, mils | thickness, mm | mg/sq. inch |
| 1 | 5.0 | 100-250 | 1.25 | 20 | 0.46 | 65 |
| 2 | 5.0 | 100-250 | 2.14 | 15 | 0.33 | 70 |

While this invention has been particularly shown and described with references to preferred embodiments thereof, it will be understood by those skilled in the art that various changes in form and details may be made therein without departing from the scope of the invention encompassed by the appended claims.

1. An acid addition salt of a basic therapeutic agent wherein the acid is represented by Formula I: 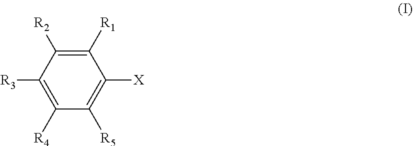
wherein R₁, R₂, R₃, R₄ and R₅ are each independently hydrogen, halogen, C₁-C₁₂-alkyl or halo-C₁-C₁₂-alkyl; and X is —SO₃H, —C(O)OH or —PO(OR₆)(OH), where R₆ is hydrogen or C₁-C₆-alkyl; provided that at least one of R₁, R₂, R₃, R₄ and R₅ is not hydrogen and further provided that the compounds of Formula I do not include 4-methylbenzenesulfonic acid.

2. (canceled)

3. (canceled)

4. The acid addition salt of claim 1, wherein: 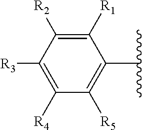
is pentafluorophenyl, pentachlorophenyl or pentabromophenyl.

5. The acid addition salt of claim 1, wherein at least one of R₁ to R₅ is a C₃-C₁₂ alkyl group.

6. (canceled)

7. (canceled)

8. The acid addition salt of claim 1, wherein at least one of R₁ to R₅ is a perhalo-C₃-C₁₂-alkyl group.

9. (canceled)

10. The acid addition salt of claim 1, wherein at least one of R₁ to R₅ is a halogen and at least of the others is an alkyl or haloalkyl group.

11. (canceled)

12. The acid addition salt of claim 1, wherein the compound of Formula I is selected from the group consisting of 4-fluoro-2,6-dimethylbenzene-1-sulfonic acid; 3,5-difluoro-4-methylbenzene-1-sulfonic acid; 2,4,6-trimethylbenzene-1-sulfonic acid; 4-chloro-2,6-dimethylbenzene-1-sulfonic acid; 2,6-dimethyl-4-(trifluoromethyl)benzene-1-sulfonic acid; 4-bromo-2,6-dimethylbenzene-1-sulfonic acid; 2,6-dimethyl-4-(trichloromethyl)benzene-1-sulfonic acid; 4-iodo-2,6-dimethylbenzene-1-sulfonic acid; 3,5-dichloro-4-methylbenzene-1-sulfonic acid; 2,4,6-trichloro-3,5-dimethylbenzene-1-sulfonic acid; 3,5-dibromo-4-methylbenzene-1-sulfonic acid; 3,5-diiodo-4-methylbenzene-1-sulfonic acid; 4-bromo-3,5-bis(trifluoromethyl)benzene-1-sulfonic acid; 2,3,4,5,6-pentachlorobenzene-1-sulfonic acid; 2,3,4,5,6-pentafluorobenzene-1-sulfonic acid; 2,4,6-triethylbenzene-1-sulfonic acid; 4-bromo-3,5-bis(trichloromethyl)benzene-1-sulfonic acid; 2,6-dimethyl-4-(tribromomethyl)benzene-1-sulfonic acid; 2,4,6-tri(trichloromethyl)benzene-1-sulfonic acid; 2,4,6-tri(trifluoromethyl)benzene-1-sulfonic acid; 4-iodo-3,5-bis(trifluoromethyl)benzene-1-sulfonic acid; 2,6-dimethyl-4-(triiodomethyl)benzene-1-sulfonic acid; 2,4,6-tripropylbenzene-1-sulfonic acid; 2,4,6-tri(tribromomethyl)benzene-1-sulfonic acid; and 2,4,6-tri(trifluoromethyl)benzene-1-sulfonic acid.

13. An acid addition salt of a basic therapeutic agent wherein the acid is of Formula II,
Ar—X (II),
wherein Ar is an optionally substituted polycyclic aryl group and X is —SO₃H, —C(O)OH or —PO₃H.

14. (canceled)

15. The acid addition salt of claim 12, wherein the number of substituents is 0 to 4 and the substituents are independently selected from alkyl, haloalkyl and halogen.

16.-18. (canceled)

19. A salt represented by Formula III:
B(H)ₘ₊ₙYₘXₙ (III)
wherein:
B is a basic drug;
Y is 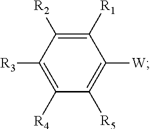
R₁, R₂, R₃, R₄ and R₅ are each independently hydrogen, halogen, C₁-C₁₂-alkyl or halo-C₁-C₁₂-alkyl;
W is SO₃⁻, C(O)O— or —P(O)(OR₆)O⁻;
R₆ is hydrogen or C₁-C₆-alkyl;
X is a pharmaceutically acceptable monoanion other than Y; and
m+n is the number of basic functional groups in B, wherein m is at least 1;
provided that at least one of R₁, R₂, R₃, R₄ and R₅ is not hydrogen; and further provided that 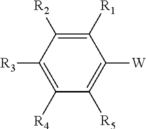
is not 4-methylbenzenesulfonate.

20. The salt of claim 19, wherein Y is selected from the group consisting of 4-fluoro-2,6-dimethylbenzene-1-sulfonate; naphthalene-1-sulfonate; 3,5-difluoro-4-methylbenzene-1-sulfonate; 2,4,6-trimethylbenzene-1-sulfonate; [1,1′-biphenyl]-4-sulfonate; 4-chloro-2,6-dimethylbenzene-1-sulfonate; 5-chloronaphthalene-1-sulfonate; 2,6-dimethyl-4-(trifluoromethyl)benzene-1-sulfonate; 4-bromo-2,6-dimethylbenzene-1-sulfonate; 5-bromonaphthalene-1-sulfonate; 2,6-dimethyl-4-(trichloromethyl)benzene-1-sulfonate; 4-iodo-2,6-dimethylbenzene-1-sulfonate; 4′-chloro[1,1′-biphenyl]-4-sulfonate; 5-iodonaphthalene-1-sulfonate; 3,5-dichloro-4-methylbenzene-1-sulfonate; 2,4,6-trichloro-3,5-dimethylbenzene-1-sulfonate; 4′-bromo[1,1′-biphenyl]-4-sulfonate; 4′-iodo[1,1′-biphenyl]-4-sulfonate; 3,5-dibromo-4-methylbenzene-1-sulfonate; 3,5-diiodo-4-methylbenzene-1-sulfonate; 4-bromo-3,5-bis(trifluoromethyl)benzene-1-sulfonate; 2,3,4,5,6-pentachlorobenzene-1-sulfonate; 2,3,4,5,6-pentafluorobenzene-1-sulfonate; 2,4,6-triethylbenzene-1-sulfonate; 4-bromo-3,5-bis(trichloromethyl)benzene-1-sulfonate; 2,6-dimethyl-4-(tribromomethyl)benzene-1-sulfonate; 2,4,6-tri(trichloromethyl)benzene-1-sulfonate; 2,4,6-tri(trifluoromethyl)benzene-1-sulfonate; 4-iodo-3,5-bis(trifluoromethyl)benzene-1-sulfonate; 2,6-dimethyl-4-(triiodomethyl)benzene-1-sulfonate; 2,4,6-tripropylbenzene-1-sulfonate; 2,4,6-tri(tribromomethyl)benzene-1-sulfonate;
and 2,4,6-tri(trifluoromethyl)benzene-1-sulfonate.

21. (canceled)

22. (canceled)

23. The salt of claim 20, wherein B is a caine anesthetic.

24. (canceled)

25. The salt of claim 23, wherein the caine anesthetic is lidocaine, bupivacaine or ropivacaine.

26. (canceled)

27. A pharmaceutical composition comprising the acid addition salt of claim 23, and a pharmaceutically acceptable excipient or carrier.

28. The pharmaceutical composition of claim 27, comprising particles of the salt.

29. (canceled)

30. A method of treating pain in a subject in need thereof, comprising administering to the subject a therapeutically effective amount of a pharmaceutical composition of claim 23.

31. (canceled)

32. (canceled)

33. A salt represented by Formula III:
B(H)ₘ₊ₙYₘXₙ (III)
wherein:
B is a basic drug;
Y is Ar—W;
Ar is an optionally substituted polycyclic aryl group;
W is SO₃⁻, C(O)O— or —PO₃⁻;
X is a pharmaceutically acceptable monoanion other than Y; and
m+n is the number of basic functional groups in B, wherein m is at least 1.

34. The salt of claim 33, wherein Ar—W is selected from the group consisting of naphthalene-1-sulfonate; [1,1′-biphenyl]-4-sulfonate; 5-chloronaphthalene-1-sulfonate; 5-bromonaphthalene-1-sulfonate; 4′-chloro[1,1′-biphenyl]-4-sulfonate; 5-iodonaphthalene-1-sulfonate; 4′-bromo[1,1′-biphenyl]-4-sulfonate; and 4′-iodo[1,1′-biphenyl]-4-sulfonate.

35. (canceled)

36. (canceled)

37. The salt of claim 33, wherein B is a caine anesthetic.

38. (canceled)

39. (canceled)

40. A pharmaceutical composition comprising the salt of claim 37, and a pharmaceutically acceptable excipient or carrier.

41. (canceled)

42. (canceled)

43. A method of treating pain in a subject in need thereof, comprising administering to the subject a therapeutically effective amount of a pharmaceutical composition of claim 40.

44. (canceled)

45. (canceled)
